(19) United States
(12) Patent Application Publication
(10) Pub. No.: US 2015/0344842 A1
(43) Pub. Date: Dec. 3, 2015

(54) METHOD FOR PRODUCTION OF DECELLULARIZED BIOLOGICAL MATERIAL AND THE DECELLULARIZED BIOLOGICAL MATERIAL PREPARED THEREFROM

(71) Applicant: Taipei Medical University, TAIPEI (TW)

(72) Inventors: MING-THAU SHEU, TAIPEI (TW); HSIU-O HO, TAIPEI (TW); YING-CHEN CHEN, TAIPEI (TW)

(73) Assignee: Taipei Medical University, TAIPEI (TW)

(21) Appl. No.: 14/292,279

(22) Filed: May 30, 2014

(51) Int. Cl. C12N 5/00

(52) U.S. Cl. CPC C12N 5/0081; C12N 2533/90; C12N 2509/00

(57) ABSTRACT

The invention provides a method for decellularization of a biological material to obtain a decellularized biological material. Compared to untreated biological material, the content of DNA of the decellularized biological material of the invention decreases to a low level and there is no significant reduction of glycosaminoglycan and collagen. The invention also provides a decellularized biological material prepared from the method of the invention and a support comprising the decellularized biological material.

### FIELD OF THE INVENTION

The invention relates to a decellularized biological material and a method of producing the decellularized biological material and its use in diverse applications. More particularly, the invention relates to a decellularized extracellular matrix (ECM) prepared by using formic acid to perform decellularization.

### BACKGROUND OF THE INVENTION

Articular cartilage allows for the relative movement of opposing joint surfaces under high loads, and cartilage defects are accompanied by persistent pain and functional limitations of the joint and are therefore considered as severe medical problems. Damaged articular cartilage displays a limited capacity of self regeneration due to the low metabolic activity and poor proliferation rates of matured chondrocytes and due to the absence of either vascular or lymphatic vessels. Cartilage transplantation has never met success due to the scarcity of autologous or allogous donor tissues; moreover, surgical techniques like drilling of subchondral bone, abrasion arthroplasty, or mosaicplasty have only produced fibrocartilage with short-term relief, but develops progressive symptoms when the repair tissue fails. Given the severely impaired quality of life, the limitations of current treatments, and the steadily growing number of patients, the urgency is high for exploring alternative treatment strategies of degenerative cartilage disease; thus, this has been the major motivation of cartilage tissue engineering.

Cartilage is organized by a unique extracellular matrix (ECM) produced and maintained by chondrocytes. The ECM, including collagen fibrils, predominantly type II collagen and link proteins such as proteoglycans, aggrecan, glycosaminoglycan (GAG) and hyaluronic acid, has a structural function, contributes to the mechanical property of cartilage, has a feedback regulatory role on chondrocyte activities, and also characterizes the phenotype of the chondrocytes. Several natural and synthetic scaffolds have been tested in animal models for cartilage engineering. Natural scaffolds interact with cells via surface receptors and regulate signal communication, but they may be inferior mechanically and subject to variable enzymatic host degradation. Synthetic polymers are more controllable and predictable through modifying mechanical and chemical characteristics. However, unless specifically incorporated, synthetic polymers do not benefit from direct cell-scaffold interactions. Thus, an alternative approach is the use of decellularized tissue.

The decellularized scaffolds not only serve as a supporting material but also regulate cellular functions, such as cell adhesion, proliferation, ECM secretion, and tissue regeneration by various existing bioactive molecules, such as, ECM, growth factors and cytokines. Components of the ECM are generally conserved among species and tolerated well even by xenogeneic recipients. However, xenogeneic or allogeneic cellular antigens are recognized as foreign bodies by the host and hence may induce severe inflammations or immune-mediated rejections. Removal of cells reduces the risk of immune response so that the xenogeneic acellular ECM scaffolds could be clinically applied, but it varies widely depending on the type of tissue, species of origin, and the decellularization methods.

WO 2008/146956 relates to a method for preparing a cell-derived ECM scaffold to which chondrocytes or stem cells are attached, wherein a detergent, an organophosphorus compound or a surfactant is used to achieve decellularization. US 2012/0064043 A1 provides a method of producing a meniscus scaffold comprising decellularizing a meniscus tissue with an oxidant and detergent simultaneously to remove extraneous material and increase the pore size and porosity therein and applying mechanical stress to the tissue in a bioreactor system. US 20140038290 A1 discloses an extracellular matrix comprising a modified polysaccharide consisting of repeating disaccharide units whereby in at least 11% of the disaccharide units, one primary alcohol group is oxidized into a carboxylic acid. US 20140023723 A1 discloses a method for producing a composition comprising a decellularized ECM, wherein the cells are removed from the tissue culture substrate by treatment with 0.5% Triton X-100 in 20 mM ammonium hydroxide in phosphate buffered saline (PBS) for 5 minutes at 37° C. to form a tissue culture substrate coated with DM.

Elder et al. evaluated the effects of different decellularization treatments (sodium dodecyl sulfate [SDS], tributyl phosphate, Triton X-100, and hypotonic followed by hypertonic solution) on articular cartilage, and found that treatment with 2% SDS for 1 or 2 hours significantly reduced the DNA content, and maintained the biochemical and biomechanical properties (Elder B D, Eleswarapu S V, Athanasiou K A 2009. Extraction techniques for the decellularization of tissue engineered articular cartilage constructs. Biomaterials 30(22):3749-3756). Furthermore, SDS treated for 6 or 8 hours resulted in complete elimination of cell nuclei, but the content of GAG and compressive properties were simultaneously significantly decreased. Thomas et al. (Tissue Engineering: Part A, Volume 14, Number 4, 2008, pp. 505-518) decellularizes menisci by exposure to several freeze-thaw cycles, incubation in hypotonic Tris buffer, 0.1% SDS in hypotonic buffer plus protease inhibitors, nucleases and hypertonic buffer followed by disinfection with peracetic acid. The results show the retention of the biomechanical properties, major structural proteins and no expression of the major xenogeneic epitope, galactose-α-1,3-galactose, but a 59.4% loss of GAG, unfortunately. Kheir et al. (Journal of Biomedical Materials Research A, November 2011, Vol. 99A, Issue 2, pp. 283-294) applied freeze/thaw cycles; followed by cyclic incubation in hypotonic tris buffer and 0.1% (w/v) sodium dodecyl sulfate in hypotonic buffer plus, protease inhibitors and nucleases (RNase and DNase) and then disinfected using 0.1% (v/v) peracetic acid. The decellularization process had minimal effect on the collagen content of the cartilage. However, there was a significant reduction (98% loss) in the glycosaminoglycan content in the decellularized tissues.

Acid treatments have been used in decellularization protocols to solubilize the cytoplasmic component of the cells as well as remove nucleic acids (Crapo P M, Gilbert T W, Badylak S F 2011. An overview of tissue and whole organ decellularization processes. Biomaterials 32(12):3233-3243). However, some acids remove cells from a biological material while simultaneously damaging biological functions of the biological material. For example, GAG and collagen are significantly decreased (Xiaochao Dong et al., Mater Med (2009) 20:2327-2336).

Although a number of decellularization treatments have been disclosed, there is still a need to develop a decellularization method to obtain a decellularized ECM having satisfied decellularization and strength which maintain functionally equivalent levels of GAG and collagen to those of the untreated biological material.

### SUMMARY OF THE INVENTION

The invention provides a method for decellularizing a biological material to obtain a decellularized biological material such as ECM, tissue, and organ. Compared to untreated biological material, the DNA content of the decellularized ECM of the invention decreased to a low level and there was no significant reduction of GAG and collagen.

In one aspect, the invention provides a method for production of a decellularized biological material, comprising providing an untreated biological material having cells and treating the biological material with a formic acid solution at a concentration effective to remove cellular and nuclear material from the biological material while maintaining higher than about 85% glycosaminoglycan (GAG) compared to the untreated biological material. In one embodiment, the method further maintains higher than about 75% collagen compared to the untreated biological material. Preferably, the decellularized biological material is a decellularized ECM, tissue or organ.

In some embodiments, the biological material to be treated by the method of the invention is skin, heart valve, pericardia, blood vessel, spinal cord, trachea, bladder, ligmant, cartilage, meniscus, disc, bone, dura mater, small intestine submucosa, spinal meninges, kindey, liver, lung, or nerve. Preferably, the tissue and organ treated by the method of the invention is articular cartilage, meniscus, disc tissue, or bone tissue.

In some embodiments, the concentration of the formic acid in the solution ranges from about 10% (w/w) to about 100% (w/w), about 15% (w/w) to about 100% (w/w), about 20% (w/w) to about 100% (w/w), about 30% (w/w) to about 100% (w/w), about 40% (w/w) to about 100% (w/w), about 50% (w/w) to about 100% (w/w), about 60% (w/w) to about 100% (w/w), about 70% (w/w) to about 100% (w/w), about 80% (w/w) to about 100% (w/w), about 90% (w/w) to about 100% (w/w), about 60% (w/w) to about 99% (w/w), about 70% (w/w) to about 99% (w/w), about 80% (w/w) to about 99% (w/w) or 90% (w/w) to about 99% (w/w). In one embodiment, the formic acid solution can further comprise other acids or cosolvents. In one embodiment, the biological material is treated with a formic acid solution for less than 15 hours, preferably less than 12 hours; more preferably, 2 to 12 hours. In some embodiments, the ratio of the biological material to the formic acid is about 1% (w/v) to about 5% (w/v), about 1% (w/v) to about 4% (w/v), about 1% (w/v) to about 3% (w/v), about 1% (w/v) to about 2% (w/v), about 1.5% (w/v) to about 5% (w/v), 1.5% (w/v) to about 4% (w/v) or 1.5% (w/v) to about 3% (w/v). More preferably, the ratio of the biological material to the formic acid is about 2%.

In one embodiment, the method of the invention removes cellular and nuclear material from the biological material while maintaining levels of GAG. Compared to the untreated biological material, the content of DNA significantly decreased and there is no obvious reduction of GAG. In one embodiment, the content of DNA decreases to less than about 5% or lower compared to the untreated biological material while maintaining higher than about 85% GAG. Preferably, the method of the invention further maintains higher than about 75% collagen.

The invention also provides a decellularized biological material, wherein the contents of DNA and GAG are less than about 5% and higher than about 85%, respectively. In one embodiment, the decellularized biological material has higher than about 75% collagen. Preferably, the decellularized biological material is a decellularized ECM, tissue or organ. In some embodiments, the decellularized biological material of the invention can be combined as part of one or more types of supports such as pharmaceutical compositions, implants, tissue regeneration scaffolds, and medical devices.

The invention also provides a method for preparation of an in vitro scaffold culture system and a method for treating a subject requiring implantation of tissue or an organ or treating a subject at a risk of implantation of tissue or an organ for prophylaxis, comprising using the decellularized biological material (preferably decellularized ECM) or scaffold of the invention.

The decellularized biological material (preferably decellularized ECM) or scaffold of the invention can be used to treat defective, diseased, damaged, injured, aged or ischemic tissues or organs which include, but are not limited to, cartilage, disc, articular cartilage, bone, dura mater, meniscus, head, neck, eyes, mouth, throat, esophagus, chest, bone, ligaments, tendons, lung, colon, rectum, stomach, prostate, pancreas, breasts, ovaries, fallopian tubes, uterus, cervix, testicles or other reproductive organs, hair follicles, skin, diaphragm, thyroid, blood, muscles, bone marrow, heart, lymph nodes, blood vessels, large intestine, small intestine, kidney, liver, pancreas, brain, spinal cord, and the central nervous system.

### BRIEF DESCRIPTION OF THE DRAWING

FIG. 1. Contents of DNA, glycosaminoglycan, collagen, and type I and II collagen of porcine menisci treated with formic acid for 2 h are compared to fresh menisci treated with PBS. Values are presented as the mean±SD (n=5). * p<0.05.

FIG. 2. Macroscopic view of a fresh porcine meniscus (left) and a decellularized one treated with formic acid for 2 h (right).

FIG. 3. Images of fresh porcine menisci (A, C, E, and G), and those treated with formic acid for 2 h (B, D, F, and H) after staining with H&E (A and B), Alcian blue (C and D), Masson's trichrome (E and F), and immunohistochemistry for type II collagen (G and H). Scale bar: 100 μm at 100× (A-H) and 50 μm at 400× (A1-H1) magnification.

FIG. 4. SEM photographs of acellular ECM scaffolds on which human chondrocytes were seeded for 0 (A), 7 (B), 14 (C), 21 (D), and 28 days (E). Scale bar: 200 μm at 300× (A-E) and 100 μm at 500× (A1-E1) magnification.

FIG. 5. Fluorescent-stained photographs of live/dead cells when human chondrocytes were cultured in acellular ECM scaffolds for 7 (A), 14 (B), 21 (C), and 28 days (D). Scale bar: 500 μm.

FIG. 6. H&E staining images of acellular ECM scaffolds onto which human chondrocytes were seeded for 7 (A), 14 (B), 21 (C), and 28 days (D). Scale bar: 100 μm at 100× (A-D) and 50 μm at 400× (A1-D1) magnification.

FIG. 7. Alcian blue staining images of acellular ECM scaffolds onto which human chondrocytes were seeded for 7 (A), 14 (B), 21 (C), and 28 days (D). Scale bar: 100 μm at 100× (A-D) and 50 μm at 400× (A1-D1) magnification.

FIG. 8. Masson's trichrome staining images of acellular ECM scaffolds onto which human chondrocytes were seeded for 7 (A), 14 (B), 21 (C), and 28 days (D). Scale bar: 100 μm at 100× (A-D) and 50 μm at 400× (A1-D1) magnification.

FIG. 9. Immunohistochemical staining of type II collagen images of acellular ECM scaffolds onto which human chondrocytes were seeded for 7 (A), 14 (B), 21 (C), and 28 days (D). Scale bar: 100 μm at 100× (A-D) and 50 μm at 400× (A1-D1) magnification.

FIG. 10. Contents of DNA (A), glycosaminoglycan (B), collagen (C), and type II collagen (D) in ECM acellular scaffolds on which human chondrocytes were cultured at different time points. Value are presented as the mean±SD (n=5).

FIG. 11. Gene expression levels of aggrecan (A), type II collagen (B), type X collagen (C), and type I collagen (D) in decellularized porcine menisci seeded with chondrocytes at different time points. Value are presented as the mean±SD (n=3).

FIG. 12. Gene expression levels of aggrecan (A), type II collagen (B), and type I collagen (C) when bone-marrow-derived human mesenchymal stem cells were cultured in a monolayer (TCPS) or on acellular ECM scaffolds (S) and fed with growth medium (M) or chondrogenic medium (C) for 14 and 21 days. Values are presented as the mean±SD (n=3).

FIG. 13. Images of ECM acellular scaffolds seeded with bone-marrow-derived human mesenchymal stem cells for 21 days and stained with Alcian blue (A), Masson's trichrome (B), and immunohistochemistry of type II collagen (C). Scale bar: 100 μm at 100×.

FIG. 14. Gene expression levels of aggrecan (A), type II collagen (B), and type I collagen (C) when bone-marrow-derived human mesenchymal stem cells were cultured in a monolayer (TCPS) or on acellular ECM scaffolds (S) and fed with growth medium (M) or chondrogenic medium (C) for 14 and 21 days. Values are presented as the mean±SD (n=3).

FIG. 15. Contents of DNA and glycosaminoglycan of porcine menisci treated with formic acid for different periods are compared to fresh menisci treated with PBS.

FIG. 16. Contents of DNA of porcine menisci treated with different concentrations of formic acid for 2 or 4 h.

FIG. 17. Contents of DNA of porcine skin following various decellularization treatments are compared to fresh skin treated with PBS. Values are presented as the mean±SD (n=3).

FIG. 18. Contents of DNA (A), collagen (B), and glycosaminoglycan (C) of porcine menisci following various decellularization treatments at different time points are compared to fresh menisci treated with PBS. Values are presented as the mean±SD (n=3).

### DETAILED DESCRIPTION OF THE INVENTION

The invention develops a method for decellularization of a biological material to obtain a decellularized biological material such as ECM, tissue, and organ; the biological material is preferably cartilage, articular cartilage, meniscus, bone or skin. The decellularization method of the invention has minimal adverse effect on the biological material (preferably ECM) to prepare acellular scaffolds. Compared to untreated biological material, the content of DNA of the decellularized biological material of the invention decreases to a low level and there is no significant reduction of GAG and collagen. The decellularized biological material of the invention not only provides temporary habitation, but also has diverse physiological functions and native environment to promote the cell proliferation and new tissue formation.

Various aspects of the invention are described in further detail in the following subsections. Unless otherwise defined, all technical and scientific terms used herein have the same meaning as is commonly understood by one of ordinary skill in the art to which this invention belongs. Although methods and materials similar or equivalent to those described herein can be used in the practice of the invention, examples of suitable methods and materials are described below. The materials, methods, and examples described herein are illustrative only and are not intended to be limiting. All publications, patent applications, patents, and other references mentioned herein are incorporated by reference in their entirety.

As used herein, the singular form “a,” “an” and “the” include singular and plural references unless the context clearly dictates otherwise. For example, the term “a cell” includes a single cell as well as a plurality of cells, including mixtures thereof.

As used herein, the term “and/or” refers to and encompasses any and all possible combinations of one or more of the associated listed items, as well as the lack of combinations when interpreted in the alternative (“or”).

As used herein, the term “about,” when referring to a measurable value such as an amount or concentration and the like, is meant to encompass variations of 20%, 10%, 5%, 1%, 0.5%, or even 0.1% of the specified amount.

As used herein, the term “GAG” means glycosaminoglycan.

As used herein, the terms “scaffold” and “matrix” interchangeably refer to a natural or synthetic structure or meshwork of structures with open porosity that is extended in space and provides mechanical or other support for the growth of living tissue, either in the body or in vitro.

As used herein, the terms “extracellular matrix” and “ECM” refer to a natural or artificial scaffolding for cell growth. Natural ECMs (ECMs found in multicellular organisms, such as mammals and humans) are complex mixtures of structural and non-structural biomolecules, including, but not limited to, collagens, elastins, laminins, glycosaminoglycans, proteoglycans, antimicrobials, chemoattractants, cytokines, and growth factors. In mammals, ECM often comprises about 90% collagen, in its various forms.

As used herein, the term “decellularize” refers to the removal of cells and their related debris from a portion of a tissue or organ, for example, from the ECM.

As used herein, the term “subject” refers to an animal, a mammal or, specifically a human patient.

As used herein, the term “treating” is meant to improve the condition of a subject by reducing, alleviating, reversing, or preventing at least one adverse effect or symptom.

In one aspect, the invention provides a method for producting a decellularized biological material, comprising providing an untreated biological material having cells and treating the biological material with a formic acid solution at a concentration effective to remove cellular and nuclear material from the biological material while maintaining higher than about 85% GAG compared to the untreated biological material. In one embodiment, the method of the invention further maintains higher than about 75% collagen compared to the untreated biological material.

In one embodiment, the biological material to be treated by the method of the invention is a tissue and an organ. Preferably, the tissue or organ is skin, heart valve, pericardia, blood vessel, spinal cord, trachea, bladder, ligament, cartilage, meniscus, disc, bone, dura mater, small intestine submucosa, spinal meninges, kidney, liver, lung, or nerve. More preferably, the tissue and organ treated by the method of the invention is an articular cartilage, a meniscus, a disc tissue, or a bone.

In one embodiment, a decellularized biological material of the invention is a decellularized ECM, decellularized tissue or decellularized organ. The tissue or organ is as mentioned above.

In one embodiment, the concentration of the formic acid in the solution ranges from about 10% (w/w) to about 100% (w/w). Preferably, the concentration of the formic acid ranges from about 10% (w/w) to about 100% (w/w), about 15% (w/w) to about 100% (w/w), about 20% (w/w) to about 100% (w/w), about 30% (w/w) to about 100% (w/w), about 40% (w/w) to about 100% (w/w), about 50% (w/w) to about 100% (w/w), about 60% (w/w) to about 100% (w/w), about 70% (w/w) to about 100% (w/w), about 80% (w/w) to about 100% (w/w), about 90% (w/w) to about 100% (w/w), about 60% (w/w) to about 99% (w/w), 70% (w/w) to about 99% (w/w), 80% (w/w) to about 99% (w/w) or 90% (w/w) to about 99% (w/w).

In one embodiment, the formic acid solution can further comprise cosolvents. Preferably, the cosolvents comprise any of glycerol, ethanol, 1-propanol, polyethylene glycol 600, or a mixture thereof.

In one embodiment, the formic acid solution can further comprise an acid solution. Preferably, the acid solutions comprise any of acetic, peracetic or citric acids, or a mixture thereof.

In one embodiment, the ratio of the biological material to the formic acid is about 1% (w/v) to about 5% (w/v), about 1% (w/v) to about 4% (w/v), about 1% (w/v) to about 3% (w/v), about 1% (w/v) to about 2% (w/v), about 1.5% (w/v) to about 5% (w/v), 1.5% (w/v) to about 4% (w/v) or 1.5% (w/v) to about 3% (w/v). More preferably, the ratio of the biological tissue to the formic acid is about 1% (w/v) to about 3% (w/v). More preferably, the ratio of the biological tissue to the formic acid is about 2% (w/v).

In one embodiment, the biological material is treated with a formic acid solution for less than 15 hours, preferably, less than 12 hours. Preferably, the treatment time is less than any of 1 to about 14 hours. More preferably, the treatment time is from about 1 hour to about 14 hours, about 1 hour to about 13 hours, about 1 hour to about 12 hours, about 1 hour to about 11 hours, about 1 hour to about 10 hours, about 1 hour to about 9 hours, about 1 hour to about 8 hours, about 1 hour to about 7 hours, about 1 hour to about 6 hours, about 1 hour to about 5 hours, about 1 hour to about 4 hours, about 1 hour to about 3 hours, about 1 hour to about 2 hours, about 2 hours to about 15 hours, about 2 hours to about 14 hours, about 2 hours to about 13 hours, about 2 hours to about 12 hours, about 2 hours to about 11 hours, about 2 hours to about 10 hours, about 2 hours to about 9 hours, about 2 hours to about 8 hours, about 2 hours to about 7 hours, about 2 hours to about 6 hours, about 2 hours to about 5 hours, about 2 hours to about 4 hours or about 2 hours to about 3 hours.

In one embodiment, the method of the invention removes cellular and nuclear material from the biological material while maintaining levels of GAG. In one embodiment, the the method of the invention further maintains collagen. Compared to the untreated biological material, the content of DNA significantly decreased and there is no obvious reduction of GAG and collagen. In one embodiment, the content of DNA decreases to less than about 5% or lower compared to the untreated biological material. Preferably, the content of DNA decreases to less than about 4.5%, less than about 4.1%, less than about 4.0%, less than about 3.5%, less than about 3.0%, less than about 2.5%, less than about 2.0%, less than about 1.5, less than about 1.0%, less than about 0.8%, or less than about 0.5%. In one embodiment, the content of DNA decreases to about 0.1% to about 5% compared to the untreated biological material; preferably, about 0.1% to about 5.0%, about 0.1% to about 4.5%, about 0.1% to about 4.0%, about 0.1% to about 3.0%, about 0.4% to about 5.0%, about 0.4% to about 4.5% or about 0.4% to about 4.0%.

In one embodiment, after treating the biological material by the method of the invention, higher than about 85% GAG can be maintained compared to the untreated biological material. Preferably, higher than about 85%, 87%, 89%, 90%, 91%, 92%, 93%, 94%, 95%, 96%, 97%, 98%, or 99% GAG can be maintained compared to the untreated biological material; more preferably, higher than about 90%, 91%, 92%, 93%, 94% or 95% GAG can be maintained. In one embodiment, about 85% to about 99% GAG can be maintained. Preferably, about 85% to about 98%, about 85% to about 97%, about 85% to about 96%, about 85% to about 95%, about 85% to about 94%, about 90% to about 99%, about 90% to about 98%, about 90% to about 97%, about 90% to about 96%, about 90% to about 95%, or about 90% to about 94% GAG can be maintained.

In one embodiment, higher than about 75% collagen can be maintained compared to the untreated biological material. Preferably, higher than about 75%, higher than about 77%, higher than about 78%, higher than about 79%, higher than about 80%, higher than about 82%, higher than about 84%, higher than about 86%, higher than about 88%, higher than about 90%, higher than about 92%, higher than about 94%, higher than about 96%, higher than about 97%, higher than about 98% or higher than about 99% collagen can be maintained. In one embodiment, about 75% to 99%, about 77% to 99%, about 79% to 99%, about 80% to 99%, about 80% to 95% or about 80% to 90% collagen can be maintained.

In one embodiment, before treating the biological material, the method of the invention further comprises a pretreatment step. In some embodiments, the pretreatment includes, but is not limited to, washing, disinfection, separation, homogenization, vibration, sonication, ultrasonication, perfusion, mechanical massage, pressure, pulverization, agitation or stirring processing.

In one embodiment, before treating the biological material, the method of the invention further comprises a step of physical decellularization, chemical decellularization, or the combination of the physical decellularization and the chemical decellularization. Preferably, the physical decellularization includes, but is not limited to, freezing-thawing, ultrasonication, perfusion, or physical agitation. Preferably, the chemical decellularization includes but is not limited to, acid solution, hypotonic solution, anionic, nonionic, or cationic detergent, or enzymes (DNase, RNase or trypsin).

In one embodiment, after treating the biological material, the method of the invention further comprises a washing step. The washing is a routine washing of the decellularized matrix by using a physiological buffer, phosphate buffered saline or culture media containing antibiotic, perfusion or dialysis. In one embodiment, the step of washing is conducted for half a day to seven days or longer.

In a further embodiment, after treating the biological material, the method of the invention further comprises a step of forming the decellularized biological material as a scaffold by mixing the decellularized biological material with/without porogen, pouring it into a mold for shaping a scaffold and then freeze-drying the shaped scaffold.

In another aspect, the invention provides a decellularized biological material, wherein the contents of DNA and GAG are less than about 5% or lower, and higher than about 85%, respectively, compared to untreated biological material. In one embodiment, the decellularized biological material maintains higher than about 75% compared to untreated biological material. The preferred contents of DNA, GAG and collagen are described herein. Preferably, the decellularized biological material is a decellularized ECM, decellularized tissue, or decellularized organ. The tissue or organ is as mentioned above.

In some aspects, a decellularized biological material (such as decellularized ECM, tissue or organ) of the invention is combined with a support. In particular, included herein are methods for formulating the decellularized biological material as part of one or more types of support such as pharmaceutical compositions, implants, tissue regeneration scaffolds, and medical devices. Preferably, the decellularized biological material is a decellularized ECM, decellularized tissue, or decellularized organ. The tissue or organ is as mentioned above.

Also included in the invention are methods for making and implanting an implant by using the decellularized biological material of the invention. The implants can be, without limitation.

In some aspects, a support is a biocompatible material such as a tissue regeneration scaffold. Preferably, the decellularized biological material is a decellularized ECM, decellularized tissue, or decellularized organ. The decellularized tissue or organ is as mentioned above. One aspect provides for the incorporation of the decellularized biological material into a biocompatible material for implantation into a subject. In one embodiment, the biocompatible material is in the form of a scaffold. The scaffold can be of natural collagen, decellularized, conditioned extracellular matrix, or synthetic polymer. In certain embodiments, the scaffold serves as a template for cell proliferation and ultimately tissue formation. In a specific embodiment, the scaffold allows the slow release of the decellularized extracellular matrix into the surrounding tissue. As the cells in the surrounding tissue begin to multiply, they fill up the scaffold and grow into three-dimensional tissue.

In some aspects, the support includes a medical device. The decellularized biological material can be used to form medical devices or prosthetic devices, which can be implanted in the subject. Preferably, the decellularized biological material is a decellularized ECM, decellularized tissue or decellularized organ. The decellularized tissue or organ is as mentioned above. In one embodiment, the decellularized biological material can be incorporated into the base material needed to make the medical or prosthetic device. In another embodiment, the decellularized biological material can be used to coat or cover the medical or prosthetic device. The medical and prosthetic devices can be inserted or implanted into the body of a patient.

In certain embodiments, the decellularized biological material can be used to treat defective, damaged, injured, diseased, aged or ischemic tissues or organs which include, but are not limited to, cartilage, disc, articular cartilage, bone, dura mater, meniscus, head, neck, eye, mouth, throat, esophagus, chest, bone, ligament, tendons, lung, colon, rectum, stomach, prostate, pancreas, breast, ovaries, fallopian tubes, uterus, cervix, testicles or other reproductive organs, hair follicles, skin, diaphragm, thyroid, blood, muscles, bone marrow, heart, lymph nodes, blood vessels, large intestine, small intestine, kidney, liver, pancreas, brain, spinal cord, and the central nervous system. Preferably, the decellularized biological material is a decellularized ECM, decellularized tissue, or decellularized organ. Preferably, the tissue or organ is skin, heart valve, pericardia, blood vessel, spinal cord, trachea, bladder, ligament, cartilage, meniscus, disc, bone, dura mater, small intestine submucosa, spinal meninges, kidney, liver, lung, or nerve.

For example, for treatment of defective, damaged, injured, diseased, aged or ischemic cartilage or skin, the decellularized biological material scaffold can be implanted into the above cartilage at a site of injury or at a site of a defect caused by disease or age, in a single surgery.

For example, for treatment of an osteochondral defect, due to its anatomical arrangement where the subchondral bone is localized directly beneath the injured cartilage and the injury is both the injury to the cartilage and to the subchondral bone or subchondral skeletal bone, it is an extension of the method for treatment of cartilage lesions described herein. The true bone defects, lesions or fractures are stand-alone injuries in the skeletal bone.

For example, articular cartilage is a unique tissue with no vascular, nerve, or lymphatic supply. The lack of vascular and lymphatic circulation may be one of the reasons why articular cartilage has such a poor intrinsic capacity to heal, except for formation of fibrous or fibrocartilaginous tissue. Unique mechanical functions of articular cartilage are never reestablished spontaneously after a significant injury, age wear or disease, such as osteoarthritis (OA). The decellularized ECM scaffold of the invention can be implanted to the defective articular cartilage for regeneration of articular cartilage.

In one aspect, the invention provides a method for preparation of an in vitro scaffold culture system, comprising (i) providing a decellularized biological material scaffold of the invention, (ii) perfusing a population of cells including stem cells, progenitor cells or partially differentiated progenitor cells capable of differentiation, or a population of cells capable of functional maturation to the decellularized biological material scaffold, and (iii) contacting the perfusion decellularized biological material scaffold and the population of cells under conditions and for a period of time that provide for recellularization of the perfusion decellularized biological material scaffold and differentiation and functional maturation of the stem or progenitor cells or functional maturation of the cells in the population. Preferably, the decellularized biological material is a decellularized ECM, decellularized tissue, or decellularized organ. The decellularized tissue or organ is as mentioned above. Preferably, the method further comprises providing a physiologically active substance capable of inducing cell differentiation to the organism. The physiologically active substance is selected from the group consisting of insulin like growth factor (IGF), fibroblast growth factor (FGF), tissue growth factor (TGF), bone morphogenetic proteins (BMP), nerve growth factor (NGF), platelet derived growth factor (PDGF) and tumor necrosis factor (TNF).

In another aspect, the invention provides a method for producting a tissue graft, comprising the steps of:

a) providing a decellularized biological material scaffold of the invention;
b) allowing cells in the organism to infiltrate the decellularized biological material scaffold;
c) incubating the tissue within the organism for a time sufficient for the cell to differentiate; and
d) providing a physiologically active substance capable of inducing differentiation of the cell.

Preferably, the decellularized biological material is a decellularized ECM, decellularized tissue, or decellularized organ. The decellularized tissue or organ is as mentioned above.

The invention uses formic acid to develop a protocol for decellularizing a biological material (preferably meniscus). The success of decellularization was determined by the absence of cells and the preservation of ECM compared to untreated biological material. A preliminary in vivo biocompatibility study of the decellularized biological material is sought to establish possible clinical utility of cartilage regeneration. For example, after seeding human chondrocytes in the acellular scaffold, the content of DNA, GAG, collagen, and type II collagen increased 10.10, 7.11, 4.24 and 5.11-fold at week 4. The increased gene expression levels of type II collagen and aggrecan further confirmed the maintenance of chondrocyte phenotype. Moreover, the scaffold effectively supported chondrogenesis when human bone-marrow-derived mesenchymal stem cells culturing with chondrogenic medium with the evidence of increased chondrogenic markers. Finally, in vivo implantation was conducted in rats to assess the biocompatibility. The acellular scaffold not only provided temporary habitation, but also had diverse physiological functions and native environment to promote the cell proliferation and new tissue formation.

The present invention is further illustrated by, but not limited to, the following Examples.

### EXAMPLES

### Example 1

### Decellularization of Menisci with Formic Acid

Menisci were harvested from the adult porcine knee joints, washed by phosphate buffered saline (PBS) and then freeze-dried (Eyela FD-5N, Tokyo, Japan). The lyophilized meniscus was finely shattered and 0.2 g meniscus was suspended in 10 mL of formic acid (>99%, Sigma-Aldrich, St. Louis, Mo., USA), for 2 hours with stirring. The suspension was then homogenized and dialysis. After the removal of the excess acid and cellular debris by dialysis, the meniscus slurry was placed into cylindrical moulds, and freeze-dried to fabricate the scaffolds. These scaffolds were sterilized by ethylene oxide and then degassed for characterization.

### Characterizations of Acellular ECM Scaffolds

### Biochemistry Analysis

To measure the amount of DNA, GAG, and collagen, samples were firstly digested with papain solutions (0.56 U/mL in 150 mM sodium chloride, 55 mM sodium citrate.2H₂O, 5 mM cysteine hydrochloride, and 5 mM sodium EDTA.2H₂O) at 60° C. for 16 hours. The content of double-stranded DNA was measured by the PicoGreen Quantification Kit (Molecular Probes, Eugene, Oreg., USA), and the previously reported value of 7.7 pg of DNA per chondrocyte was used to approximate the cell number. The fluorescence of the negative, DNA-free controls was subtracted from that of the experimental groups to account for the fluorescence of the scaffold alone. The amount of GAG was determined by a Blyscan® GAG assay kit (Biocolor, Newtonabbey, UK) according to the manufacturer's instruction. The specimen lysate was mixed with a Blyscan® dye reagent, and the GAG-dye complex was then collected by centrifugation. After the supernatant was removed and the tube drained, the dissociation reagent was added. Solution was then transferred into a 96-well plate and the absorbance against the background control was obtained at a wavelength of 656 nm by a microplate spectrophotometer. Chondroitin sulfate was used as a standard to calculate the concentrations of GAG in specimens.

The papain digestion was further acid hydrolyzed and reacted with chloramine-T and p-dimethylaminobenzaldehyde solution to determine the quantity of hydroxyproline. The content of hydroxyproline was converted to that of collagen using a mass ratio of 7.25. The optical density was detected by a microplate reader at 560 nm. A standard curve was established using a series of concentrations of hydroxyproline. The amounts of type I and II collagen were investigated by ELISA using the type I and II collagen detection kits (Chondrex Inc., Redmond, Wash., USA) as described by the supplier. Briefly, the samples were digested by pepsin solution overnight at 4° C. with gentle mixing for 48 hours and then with a pancreatic elastase solution at 4° C. overnight. The ELISA reaction optical density was read at 490 nm in a microplate reader.

### Histological and Immunohistochemical Staining

Meniscus before and after decellularization was fixed in 10% (v/v) neutral buffered formalin overnight, dehydrated through a series of ethanol and embedded in paraffin. Specimen cross-sections of 5-10 μm thickness were stained with hematoxylin and eosin (H&E) to visualize the nucleus, were stained with Alcian blue for GAG deposition, or stained with Masson's trichrome to observe collagen. Type II collagen was immunolocalized by an anti-type II collagen antibody (bs-0709r, Bioss, Mass., USA) with 1:100 dilution in TBST solution (Tris-buffered saline with Tween 20) and the signal was amplified with UltraVision Quanto Detection System (Thermo Scientific, MA, USA) for 10 mins followed by DAB for visualization per the manufacturer's description. Nuclei were counterstained with hematoxylin. Slides were scanned with ScanScop (Aperio, Calif., USA).

### In Vitro Study

### In Vitro Cytotoxicity

The sterilized scaffolds were incubated in serum containing media for 24 hours at 37° C. 150 μL of the scaffold extract was taken for the in vitro cytotoxicity test. NIH 3T3 mouse fibroblast cells were seeded onto a 96 well plate at a seeding density of 1×10⁴ cells/well and incubated overnight at 37° C. The media was replaced with either the extract or fresh medium, and further incubated for 24 hours. After the incubation period, the extract was replaced with fresh media containing alamar blue (Invitrogen Life Technologies, Carlsbad, Calif., USA) solution and again kept for another 2 hours incubation. The fluorescent intensity of the alamar blue/media mixture was measured by a microplate reader at an excitation wavelength of 560 nm and an emission wavelength of 620 nm. The value of fluorescence is proportional to the number of living cells and corresponds to the cells metabolic activity.

### In Vitro Chondrocyte Study

After a confluent cell layer was formed, human primary chondrocytes (ScienCell Research Laboratories, Carlsbad, Calif., USA) were detached using 0.025% trypsin plus EDTA in PBS, re-suspended in the supplemented culture medium (ScienCell Research Laboratories, Carlsbad, Calif., USA), and used for the in vitro experiments. Before cell seeding, the scaffolds were immersed in PBS and medium for 5 min, respectively, and the excess liquid was removed by sterile paper tissue, and then put in 96-well plates. Chondrocyte suspensions (10 μL) (1×10⁵ cells) were dropped directly into the scaffolds using a pippetman. The cell/scaffold constructs were placed in an incubator (37° C., 5% CO₂) for 2 h for cell adhesion before 0.2 ml of fresh growth medium was added, and the medium was changed every three days. Scaffolds without cells were also cultured similarly and taken as a blank and values were subtracted in all assays to negate interference.

### Live/Dead Cell Staining

At weeks 1, 2, 3, and 4, the cell/scaffold constructs were rinsed with sterile PBS and incubated with 8 μM calcein-AM and 4 μM ethidium homodimer-1 by the Live/dead assay Kit (Molecular Probes, Eugene, Oreg., USA). Sections were immediately examined with an inverted fluorescence microscope linked with a confocal imaging system using a FITC/Texas Red filter.

### Scanning Electron Microscopy (SEM)

After the cultivation over a desired period, the attachment of chondrocytes was observed by SEM. The cell/scaffold constructs were fixed with 2.5% (w/v) glutaraldehyde/PBS, washed with PBS (pH 7.4), post-fixed with 1% (w/v) osmium tetroxide, and then dehydrated using an ascending ethanol series. The samples were then critical-point dried and coated with gold before SEM observation (Hitachi-2400, Tokyo, Japan).

### Total RNA Extraction and Real-Time PCR

To quantify gene expression of aggrecan, type I, II and X collagen, total cellular RNA was extracted from chondrocytes grown on scaffolds at weeks 1, 2, 3 and 4, respectively, according to the standard TRIzol (Invitrogen Life Technologies, Carlsbad, USA) protocol. The concentration and purity of the extracted RNA was evaluated by the NanoDrop ND-1000 Spectrophotometer with UV absorbance. Only sample that exhibited a 260/280 nm ratio >1.8 was used in the following experiments. Isolated mRNA was transcribed into cDNA by the ReverTra Ace kit (Toyobo, Osaka, Japan).

Gene expression of aggrecan, type I, II, and X collagen was quantified by real-time PCR on a LightCycler 480 system (Roche Applied Science, Indianapolis, USA). Primers were designed by the Roche Universal ProbeLibrary Assay Design Center for the Human ProbeLibrary (www.roche-applied-science.com), and human ProbeLibrary probe no. 9 and 80 (Roche Applied Science, Indianapolis, USA) were used. Target genes were amplified using specific primers of aggrecan (NM_{—}001135, forward: 5′-cagatggacaccccatgc-3′ and reverse: 5′-cattccactcgcccttctc-3′), type I collagen (NM_{—}000088, forward: 5′-gccaacctggtgctaaagg-3′ and reverse: 5′-caggagcaccaacattacca-3′), type II collagen (NM_{—}001844, forward: 5′-gtgaacctggtgtctctggtc-3′ and reverse: 5′-tttccaggttttccagatc-3′), and type X collagen (NM_{—}000493, forward: 5′-agatcagaaagctgccaag-3′ and reverse: 5′-gcagcatattctcagatggattct-3′). The housekeeping gene glyceraldehyde-3-phosphatedehydrogenase (GAPDH) was used as a reference gene (NM_{—}002046, forward: 5′-acgggaagcttgtcatcaat-3′ and reverse: 5′-catcgccccacttgatttt-3′), and the expression of all the target genes was normalized to the GAPDH expression of that sample. For each reaction, a LightCycler Taqman Master kit (Roche Applied Science, Indianapolis, Ind., USA) was used with 400 nM of each primer, 0.2 μL probe, and 900 ng cDNA in a total volume of 10 μL. All amplifications were carried out in LightCycler 480 (Roche Applied Science, Indianapolis, Ind., USA) under the following conditions: preheat for 1 cycle at 95° C. for 15 min; amplification for 45 cycles: 95° C. for 10 s, 58° C. for 30 s, 72° C. for 3 s; and final cooling to 40° C. Based on the threshold cycle (C_{T}) values for each target and housekeeping gene, a comparative C_{T} method was used to quantify the relative amount of target genes.

### Chondrogenesis Bone-Marrow-Derived Human Mesenchymal Stem Cells

Bone-marrow-derived hMSCs were purchased from Texas A&M University Health Science Center (Temple, Tex., USA). hMSCs were cultured in a monolayer with hMSC growth medium (Lonza, Walkersville, Md., USA) at 37° C. with 5% CO₂ and medium changes every 3 days. After a confluent cell layer had formed, hMSCs were detached using 0.05% trypsin plus EDTA (Lonza), re-suspended in hMSC growth medium, and used for the following chondrogenic induction experiments. hMSC suspensions (10 μL; 10⁵ cells) were dropped directly into 96-well plates (tissue culture polystyrene [TCPS] group) or scaffolds in 96-well plates using a pipette. The TCPS and cell/scaffold constructs were placed in an incubator for 2 h for cell adhesion, after which 0.2 mL of fresh medium was added. For chondrogenesis, hMSC chondrogenic SingleQuots™ medium (Lonza) containing ITS+supplement, dexamethasone, ascorbate, sodium pyruvate, proline, GA-1000, L-glutamine, and transforming growth factor (TGF)-β3 was added for 14 and 21 days. Control groups were maintained with hMSC growth medium (Lonza). All TCPS and cell/scaffolds were kept in a humidified incubator at 37° C. with 5% CO₂ and medium changes every 3 days.

At predetermined time points, cells were harvested and analyzed for histology, biochemistry, and real-time PCR quantification.

### In Vivo Immunobiocompatibility Study

Fifteen rats with 8- to 10-week-old male Sprague Dawley were employed. After anesthetization by isoflurane (Abbott, Queenborough, UK) and disinfection with Betadine and 70% alcohol, each rat was subcutaneously inserted with a small plug of scaffolds, while 3 rats were treated as sham. A 1-cm incision was made on the dorsum of each rat and four subcutaneous pockets were made using blunt dissection. A scaffold was carefully placed in the respective pockets and the wound was closed with wound closure clipper, and the animals were returned to the housing facility, where they had free access to food and water. Weighing was performed once a week to check the sanitary constitution of the rats. On weeks 1, 2, and 4, rats were humanly sacrificed, and specimens with constructs were harvested and stained with H&E to evaluate if inflammatory response occurred. Because the ECM scaffolds were invisible after implantation, we excised large enough areas to ensure that the scaffolds were held in the specimens.

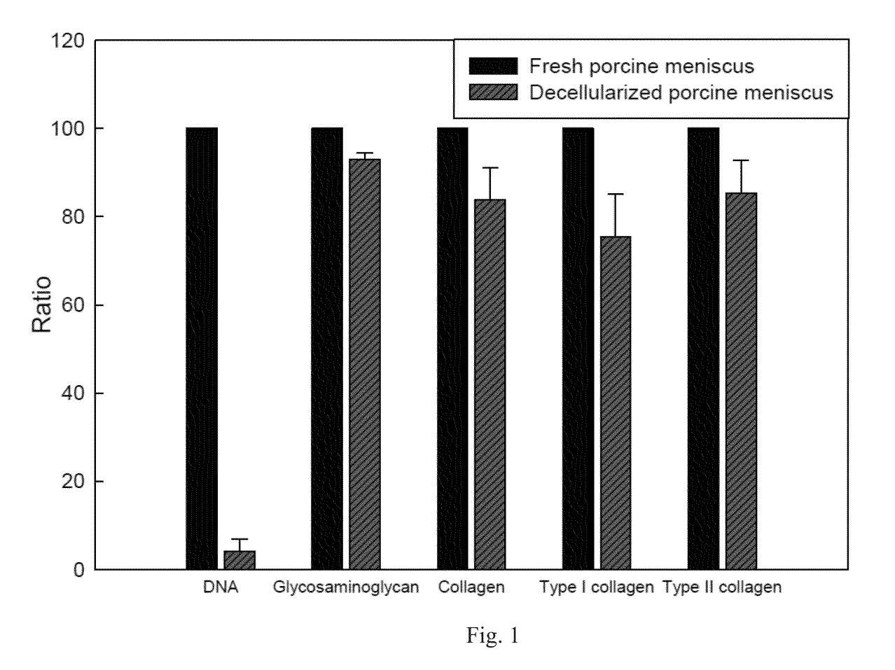

Compared to the fresh porcine menisci, FIG. 1 shows that the content of DNA significantly decreased to 4.10% (p<0.01), indicating the efficiency of the decellularization method (treatment of formic acid for 2 hours). ECM content of the scaffold was also determined by individual measurement of GAG, total collagen, and type I and II collagen. There was just 7.16% loss of GAG in content between fresh and acellular menisci (p=0.087). The amount of collagen, type I and type II collagen decreased but still remained >80% in the acellular ECM scaffold (p=0.714, 0.092 and 0.087).

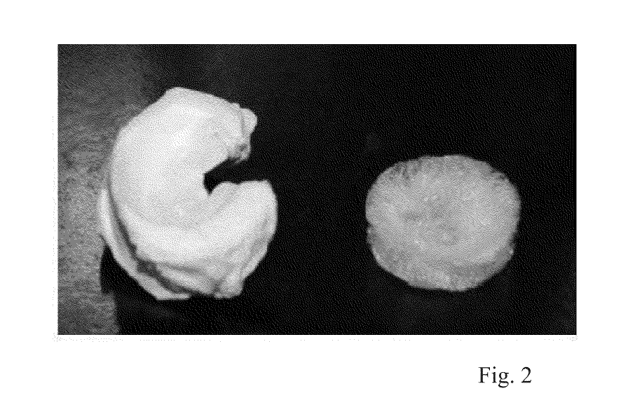

After decellularization of menisci by formic acid for 2 hours, a sponge-like porous scaffold was fabricated; a macroscopic photograph is shown in the right of FIG. 2, and the fresh compact porcine meniscus in the left of FIG. 2.

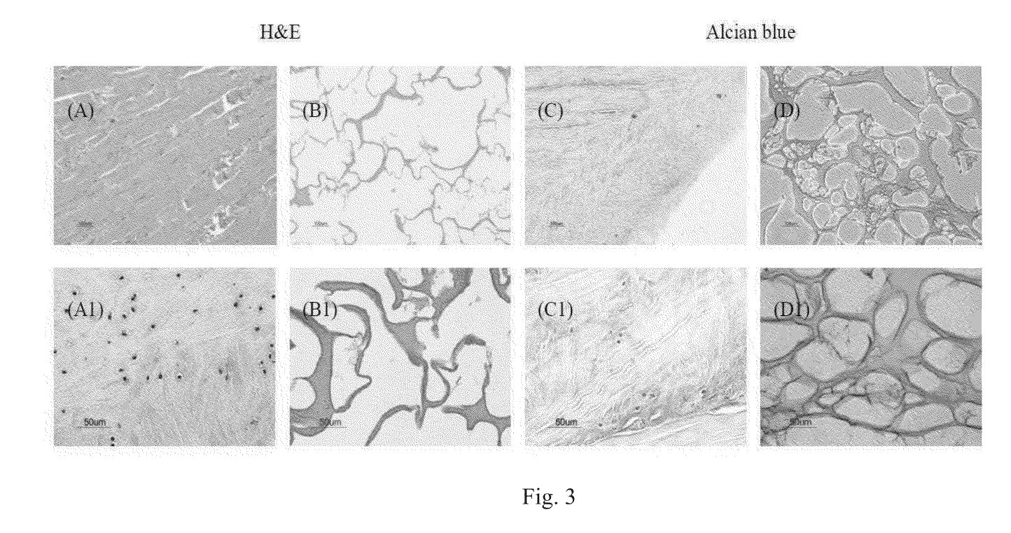

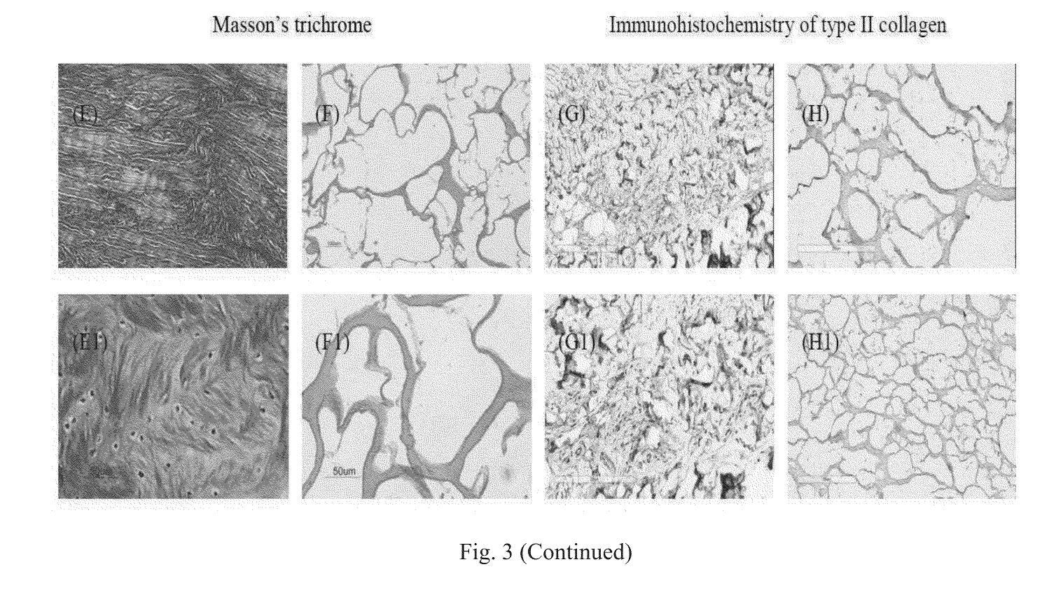

The decellularization and ECM maintenance of the scaffolds (formic acid treatment for 2 hours) were further confirmed by histology. In the fresh porcine meniscus, H&E staining (FIGS. 3 (A) and (A1)) illustrates that the chondrocytes were round and embedded within lacunae. By contrast, no cells or cell fragments were present after decellularization as shown in FIGS. 3 (B) and (B1). Alcian blue staining gave positive results on both fresh and decellularized meniscus, indicating GAG remaining after decellularization (FIGS. 3 (C), (C1), (D), and (D1)). Collagen exists as the major histoarchitecture in the fresh porcine meniscus and as well as in the decellularized scaffold through Masson's trichrome staining (FIGS. 3 (E), (E1), (F), and (F1)). FIGS. 3 (G), (G1), (H), and (H1) reveals the appearance of type II collagen after immunohistochemical examination. We demonstrated that the treatment of formic acid for 2 h was able to efficiently remove all cellular materials while minimizing any adverse effect on the composition and amount of the remaining ECM.

### In Vitro Cytotoxicity Test

The absorbance obtained from the Alamar Blue assay is directly related to the metabolic activity of the cells and inversely proportional to the toxicity of the scaffold. After two days of culture, there was no significant difference on the fluorescence value between NIH 3T3 mouse fibroblast cells seeded in monolayer (mean±SD: 33134.42±2573.10, n=6) and acellular ECM scaffold (mean±SD: 35195.14±5417.12, n=6)(p=0.167), indicating no cytotoxicity of the acellular ECM scaffold.

### Chondrocyte Attachment and Viability

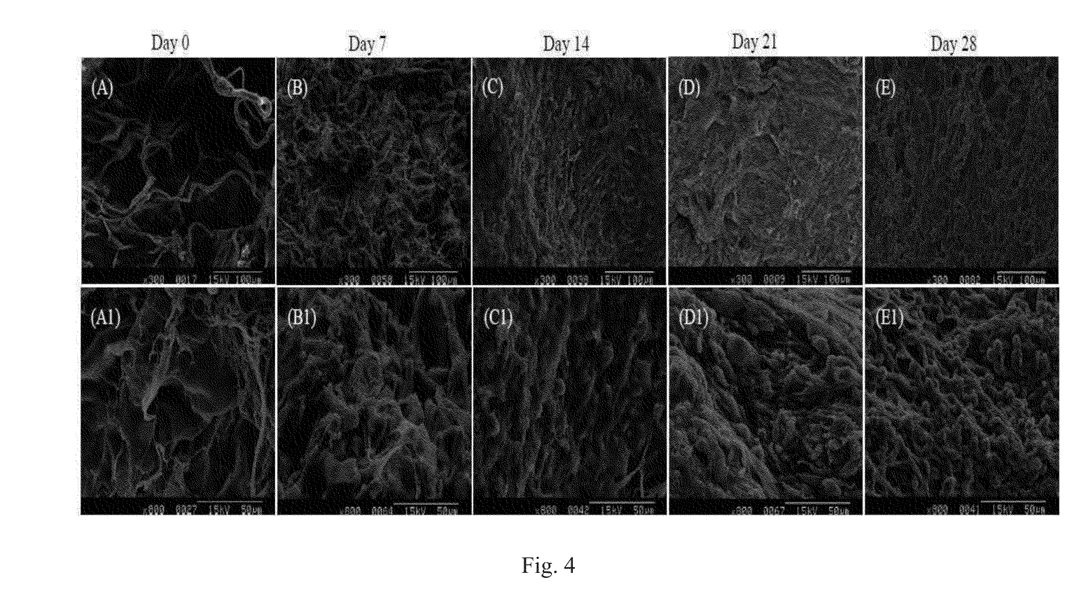

Human primary chondrocytes were seeded in the acellular ECM scaffold to evaluate the cell proliferation and ECM production. The morphology and distribution of chondrocytes in the scaffolds throughout the period of the experiment were monitored by SEM. FIG. 4 reveals chondrocytes with round or elliptic morphology attached to the scaffold well. Higher magnification showed ECM-like components on which the cells are entrapped and some cells were found to have migrated and attached to the interconnected pores. It demonstrates that chondrocytes were homogenously distributed in the porous acellular scaffold and kept their morphology even till Day 28.

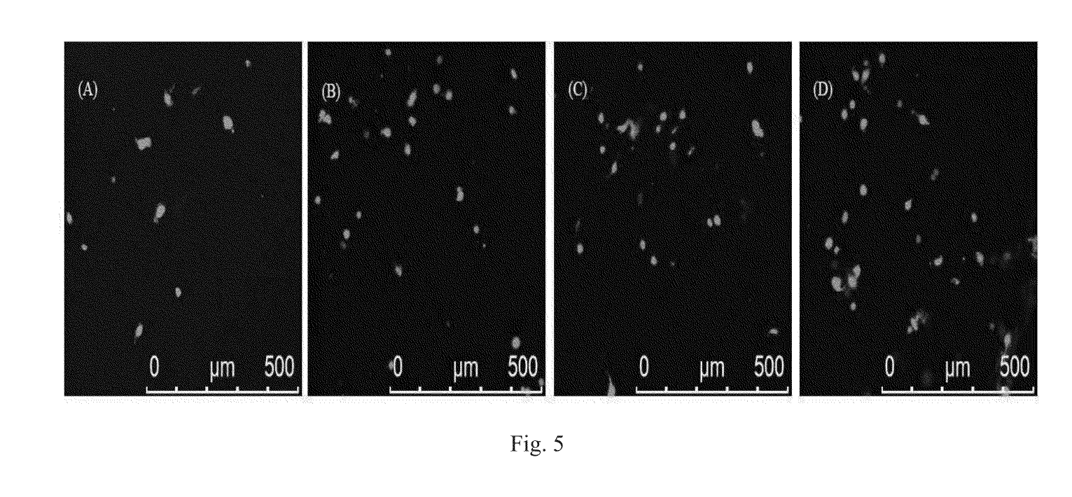

As to the live/dead cell staining (FIG. 5), calcein AM is capable of permeating the membrane of viable cells, where it is cleaved by intracellular esterases and produces green fluorescence. Ethidium bromide homodimer-1 is able to enter cells with damaged membranes and bind to fragmented nucleic acids, thereby producing red fluorescence in dead cells. On Day 7, most cells were stained with green fluorescence, except for a few cells that had died. Afterward, green living cells were observed, and very few red dead cells appeared. These results indicate that the human chondrocytes showed high viability when culturing in the acellular ECM scaffold.

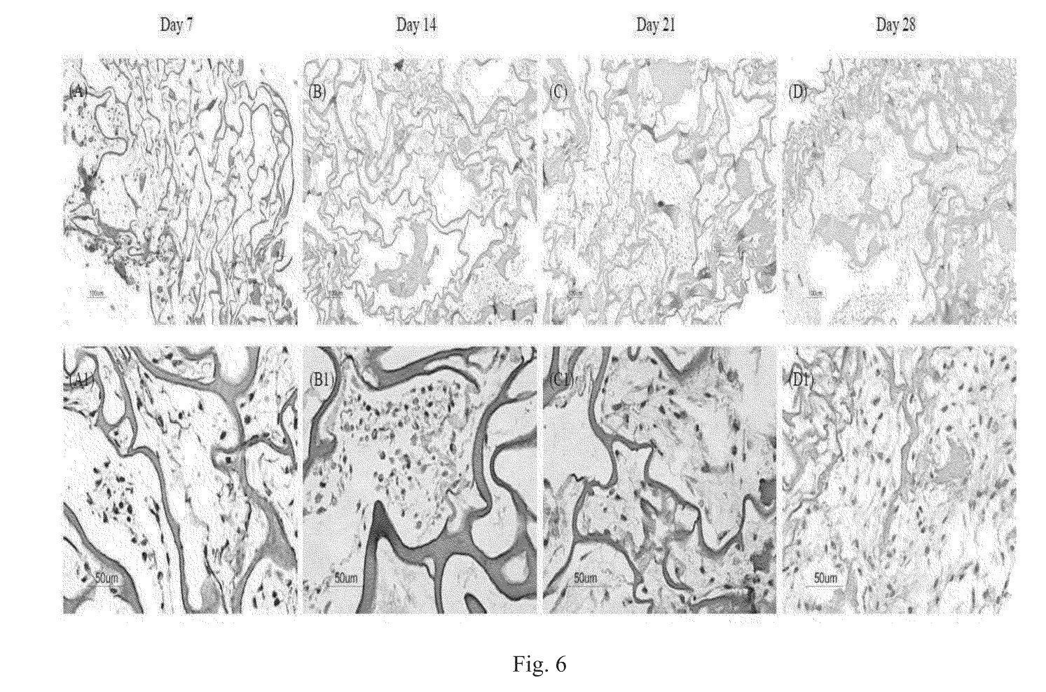

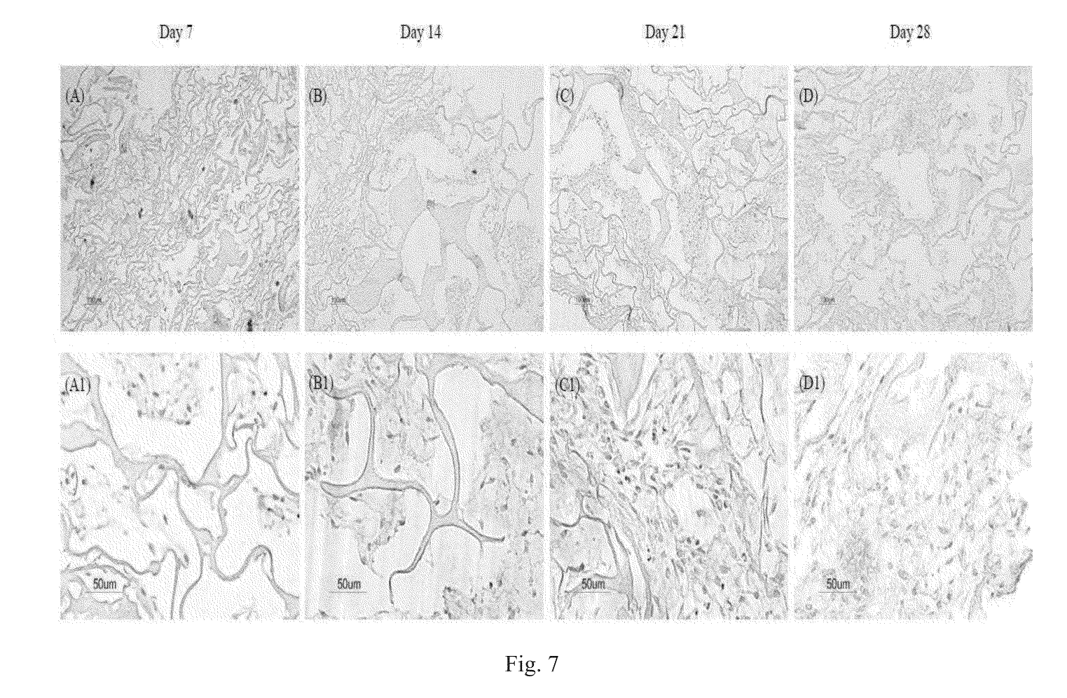

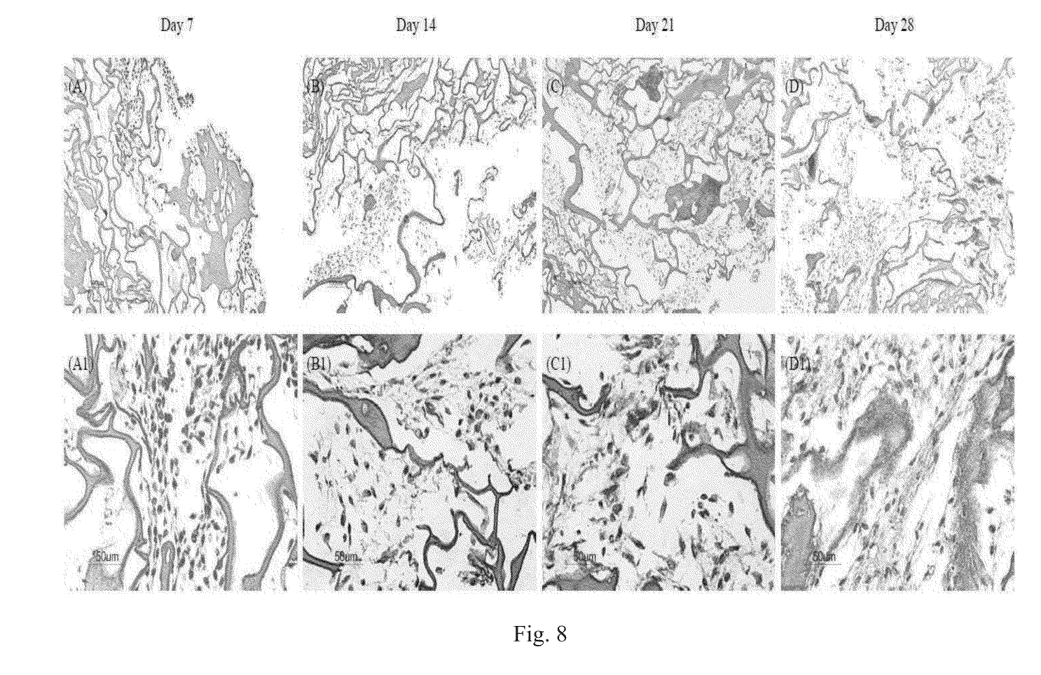

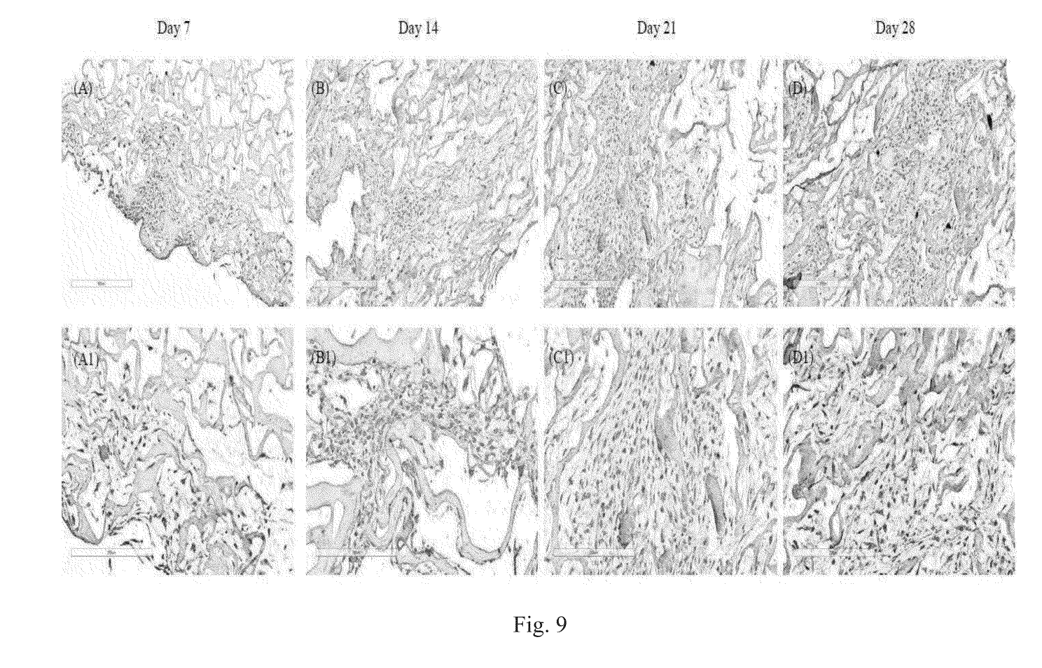

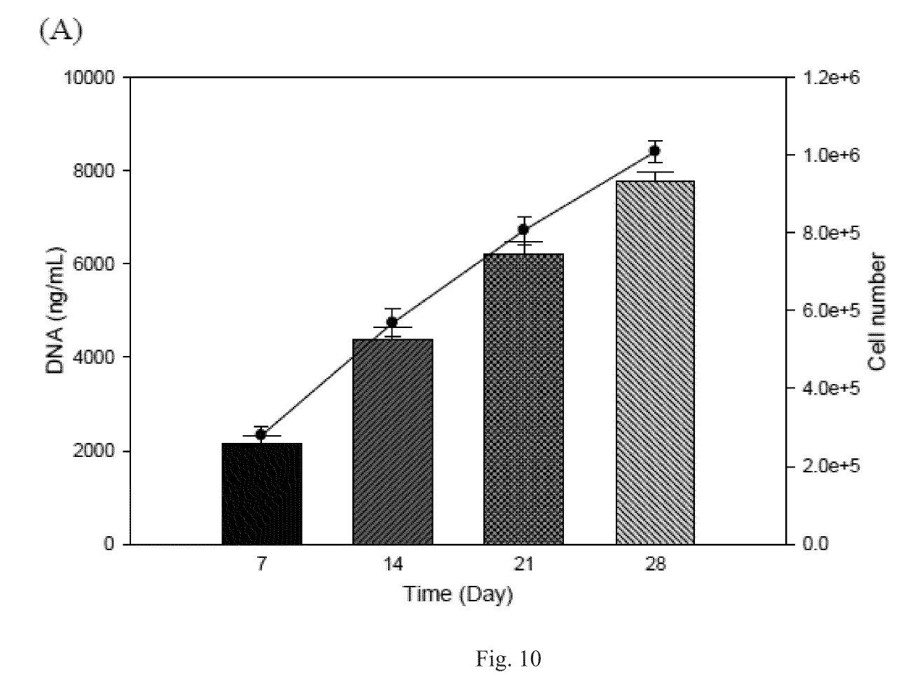

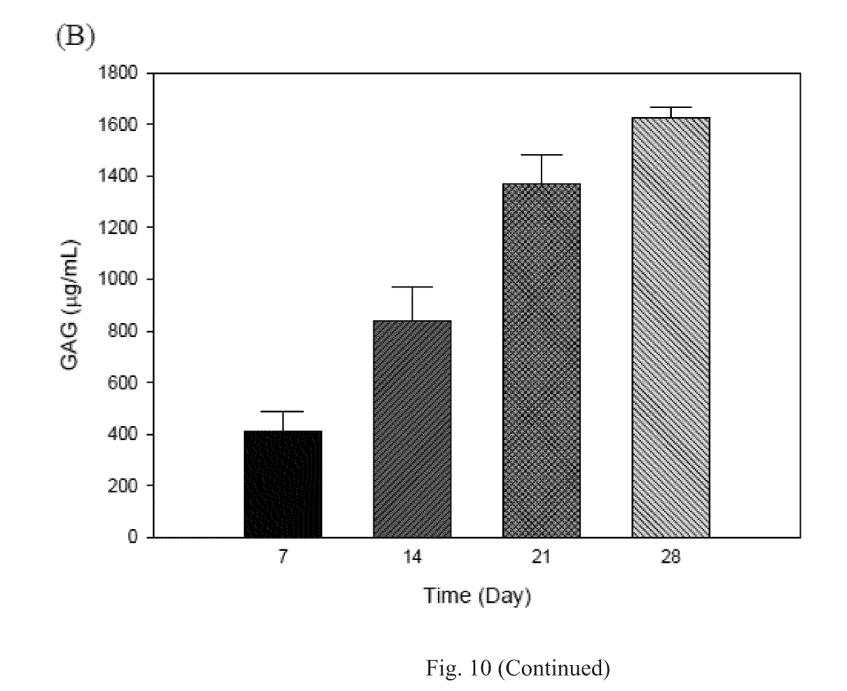

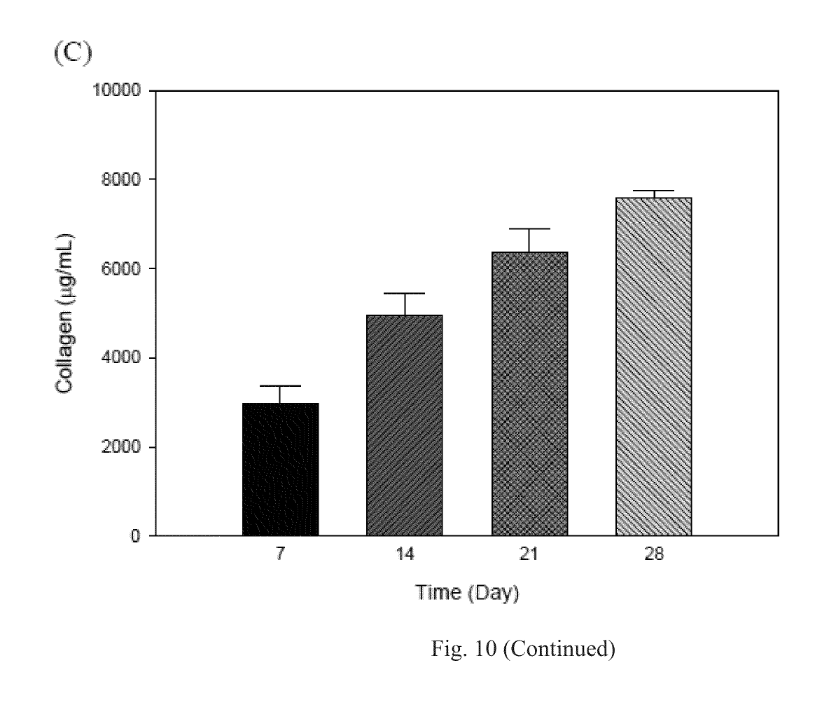

Histology and biochemistry analysis of chondrocytes to confirm the specific morphology of chondrocytes and ECM secretion occurred throughout the study period, the cell/scaffold constructs were evaluated by histological staining. H&E staining was conducted to observe the morphology, distribution and viability of cells in the acellular ECM scaffold, showing many chondrocytes with a round shape distributed evenly in FIG. 6. Significant deposition and accumulation of ECM components within the constructs was evident through Alcian blue, Masson's trichrome and immunohistochemistry staining as shown in FIG. 7-9 respectively, indicating that larger amounts of GAG, collagen, and type II collagen were being synthesized, respectively.

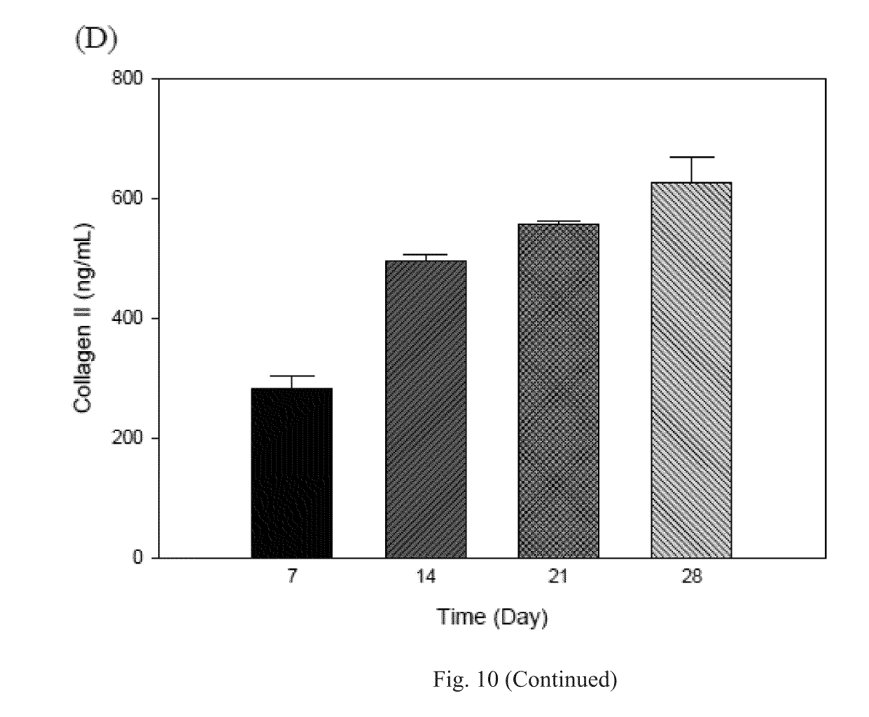

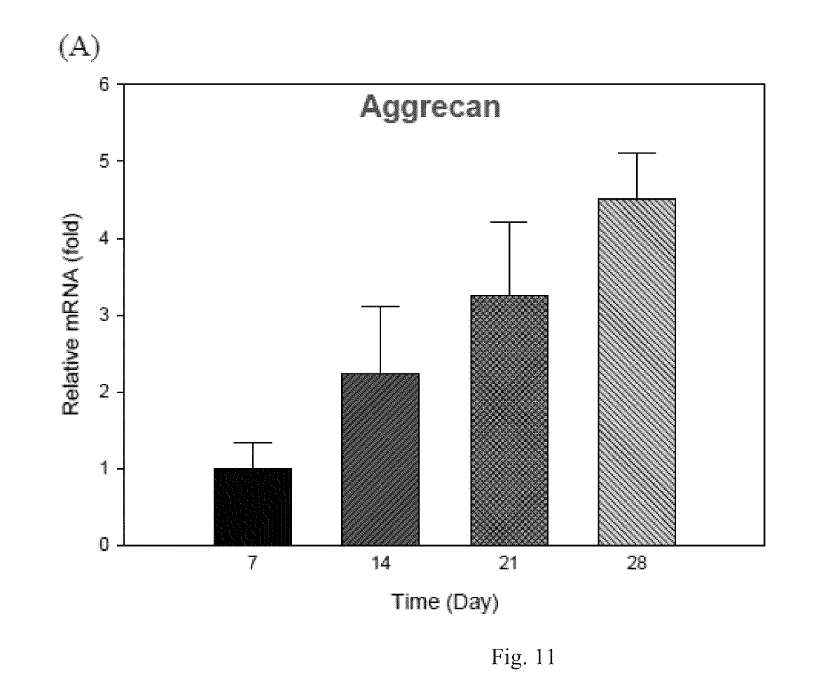

The measurement of DNA, GAG, and collagen enabled us to quantify the proliferation of chondrocytes and ECM synthesis. The content of DNA showed a 1.03, 1.89, and 2.62-fold increase after chondrocytes seeded at Day 14, 21, and 28, respectively (p<0.01) (FIG. 10 (A)), and it represents the increased cell number. The total number of cells at Day 28 reached to 10.10×10⁵, which corresponded to a 10.10-fold increase over one month. Regarding to GAG secretion, cells grown on the scaffolds showed a remarkable increase of 1.05, 3.20, 5.85 and 7.11-fold compared to blank scaffolds at Day 7, 14, 21, and 28, respectively (p<0.01) (FIG. 10 (B)). In FIG. 10 (C), the amount of collagen also showed a comparable 1.06, 2.43, 3.39 and 4.24-fold increase at different time points. As to type II collagen, the content also increased to 1.75, 3.82, 4.43 and 5.11-fold compared to blank scaffolds throughout the study period (p<0.01) (FIG. 10 (D)). By contrast, the amounts of type I collagen, a negative marker, were under the lower limit of quantitation (LLOQ, <0.08 μg/mL). The gradually increased contents of GAG, collagen, and type II collagen agreed with the result of the DNA amount; concomitantly, these findings responded to the optical microscope histological and SEM observations.

### Quantitative Real-Time PCR

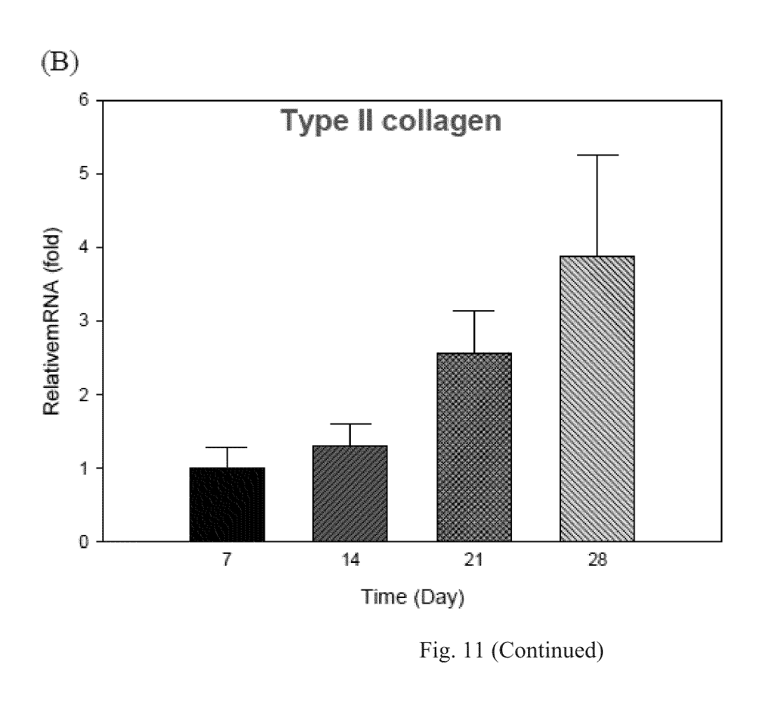

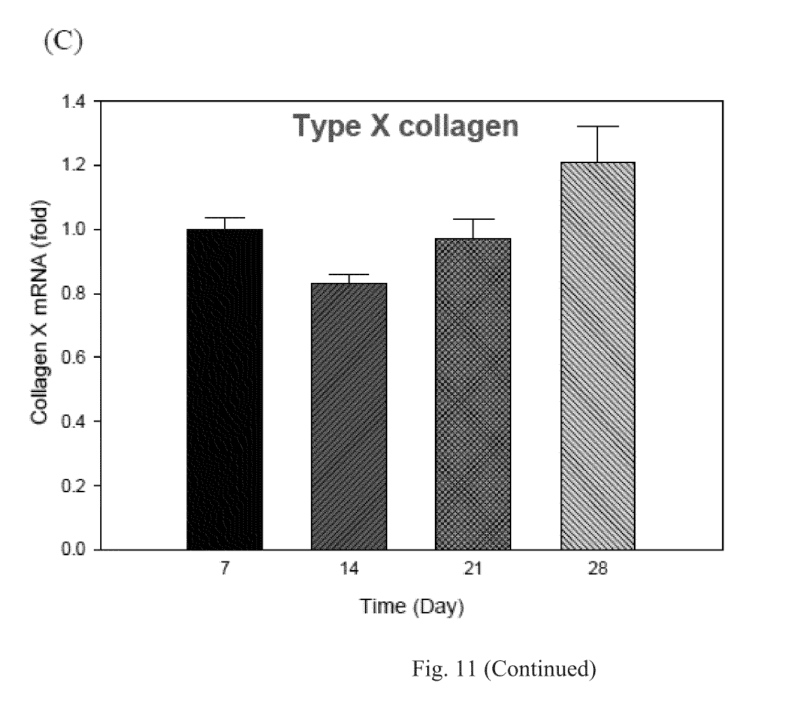

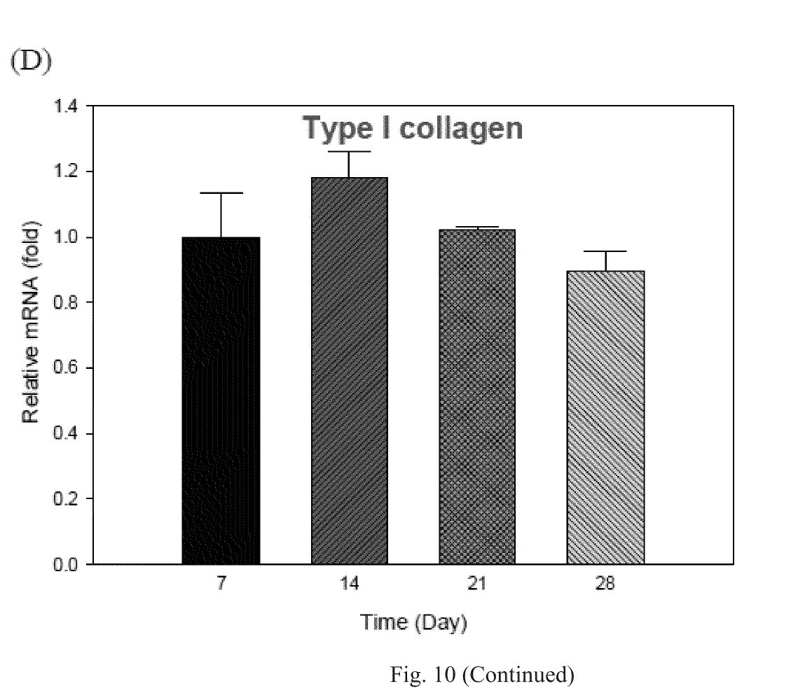

To evaluate the chondrogenic differentiation of chondrocytes seeded in the acellular ECM scaffold, the changes of the chondrogenic marker were determined by real-time PCR (FIG. 11) at Day 7, 14, 21, and 28, respectively. The two positive markers of chondrogenesis, aggrecan and type II collagen increased significantly (p<0.01). On the contrary, no significant change of the negative markers for chondrogenesis expression (type I and X collagen) was observed (p=0.155 and 0.245). The results of gene expression, together with that of histological staining and biochemical quantification indicated that human chondrocytes maintained their normal phenotypes and cartilage-like tissue was engineered when cultured in the acellular ECM scaffolds.

### Chondrogenesis of Bone-Marrow-Derived Human Mesenchymal Stem Cells

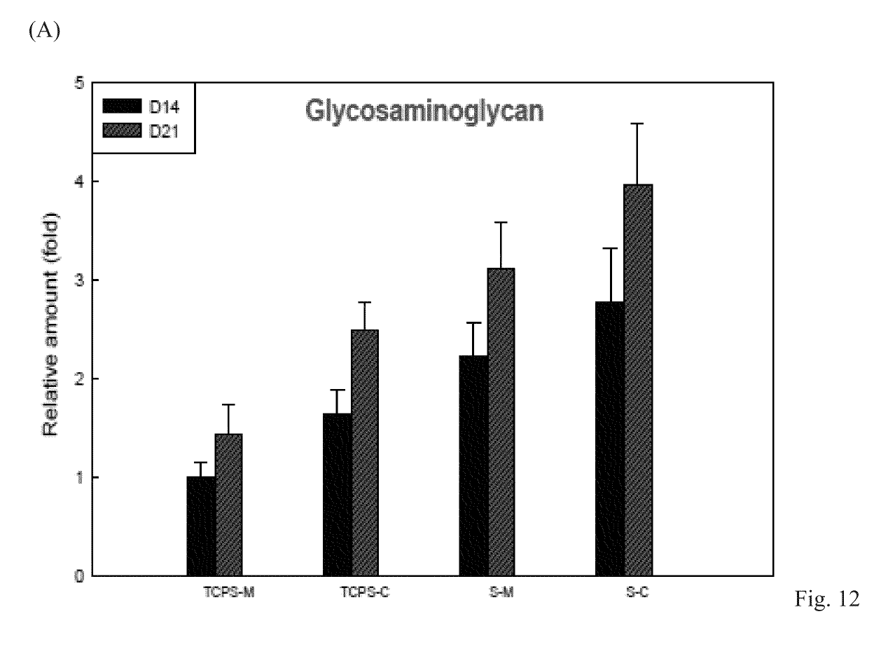

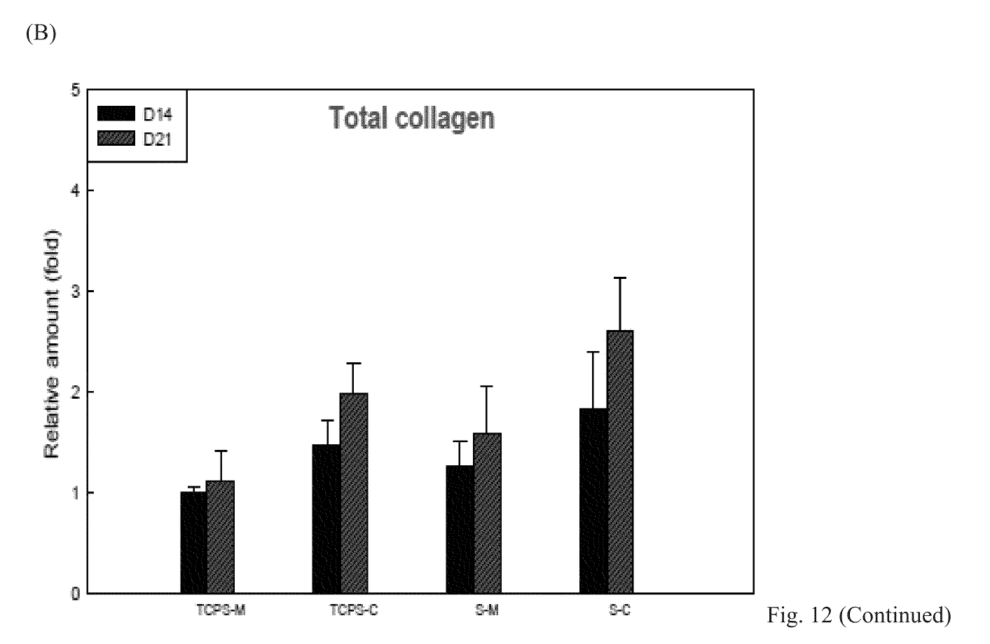

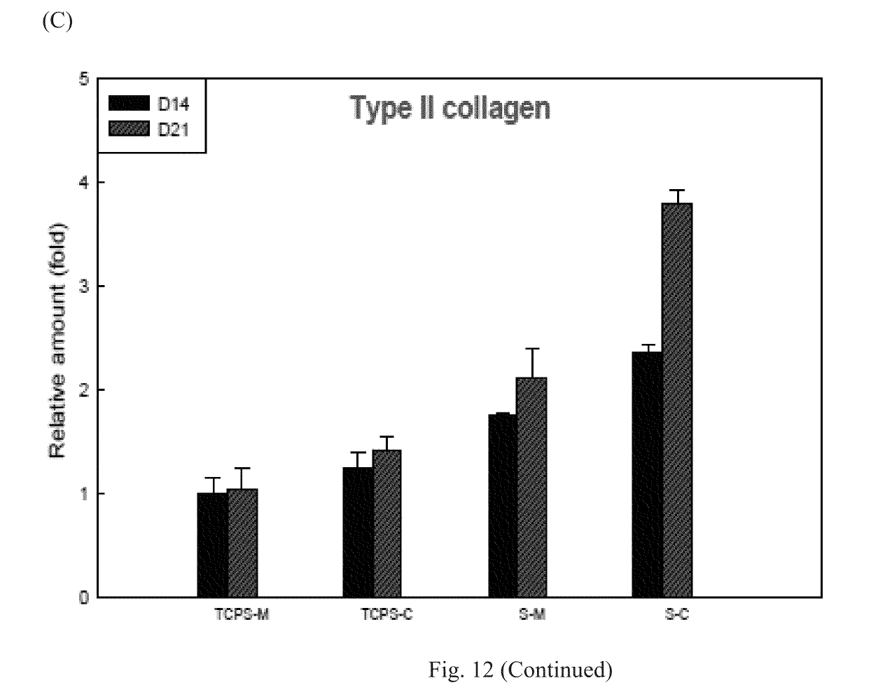

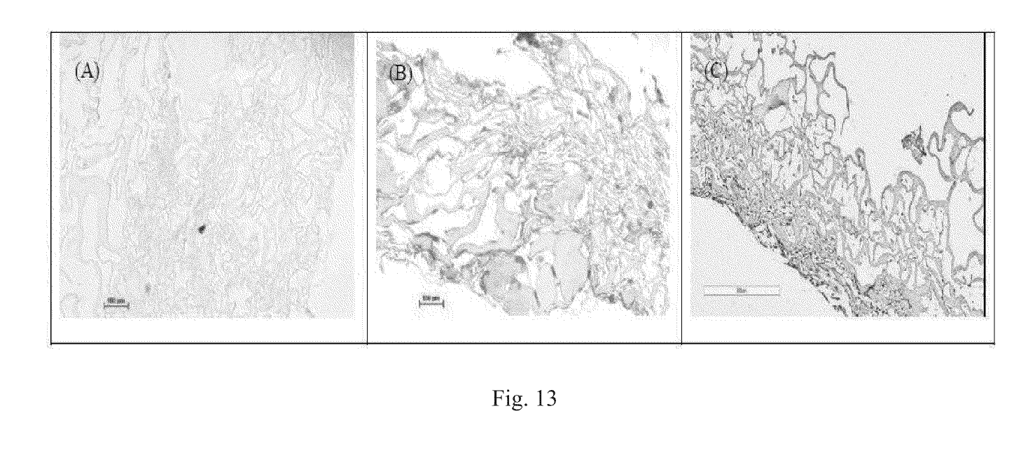

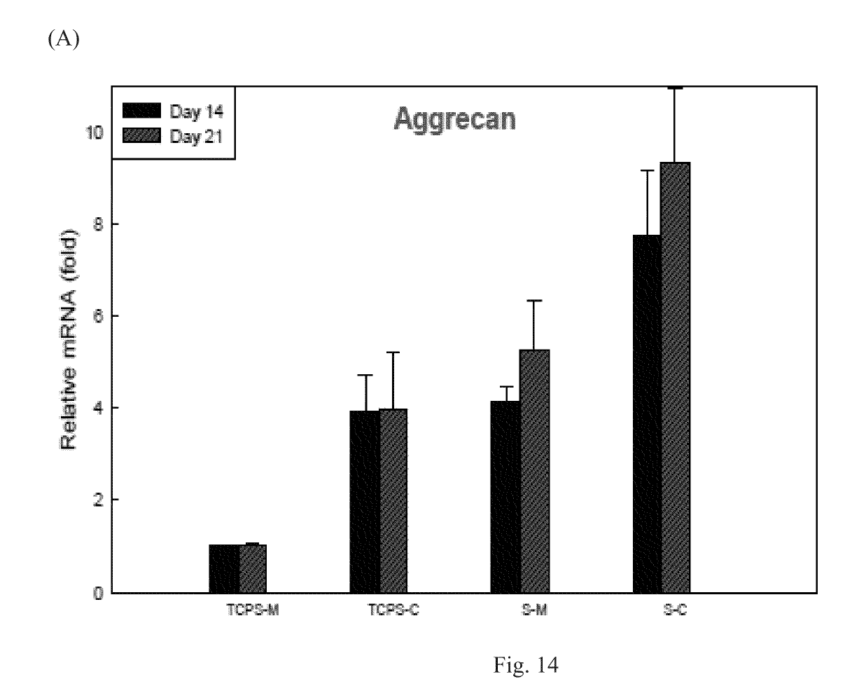

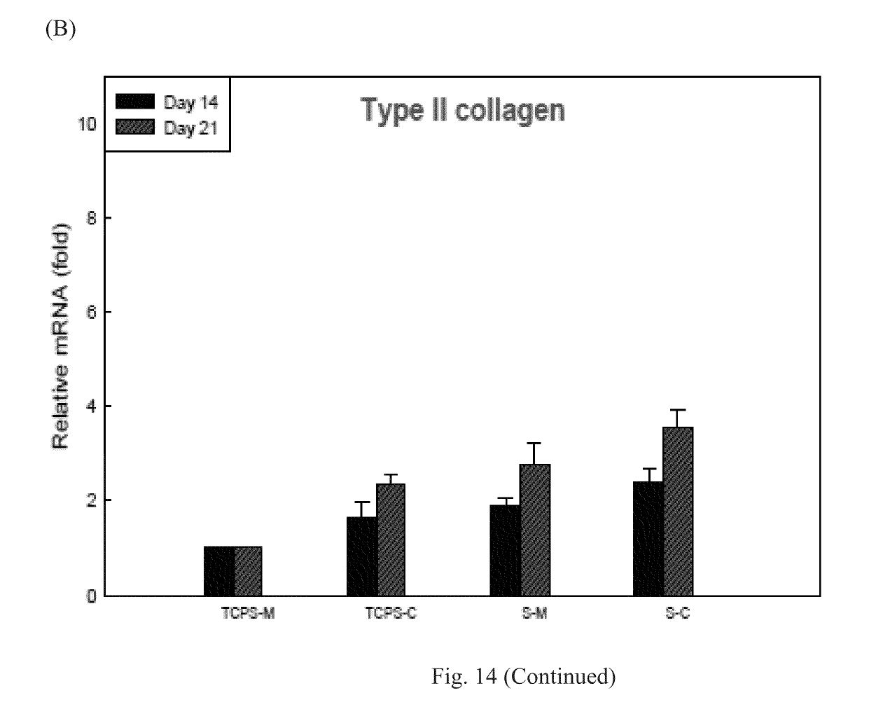

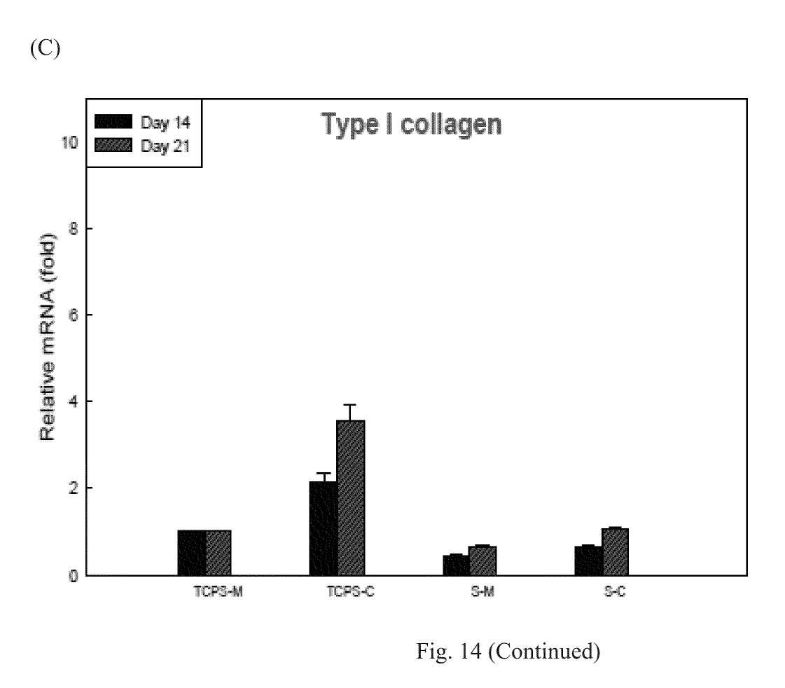

Using GAG, total collagen and type II collagen as chondrogenic differentiation markers, FIG. 12 displays the relative ratio of the average amount of ECM when hMSCs were seeded on the acellular ECM scaffolds or TCPS, and cultured with the growth or chondrogenic medium at Day 14 and 21. Compared to hMSCs seeded on the TCPS, hMSCs on the acellular ECM scaffold and cultured with chondrogenic medium synthesized higher amount of GAG, collagen and type II collagen. Histology images (FIG. 13) and the increased gene expression of aggrecan and type II collagen (FIG. 14) shows consistent results of chondrogenic differentiation, whereas a slight down-regulation of type I collagen, a marker of the chondrocyte dedifferentiation, was observed. In addition to human chondrocytes, the acellular ECM scaffold similarly provided signals to drive hMSCs toward chondrogenesis.

### In Vivo Study

The acellular ECM scaffold constructs were implanted subcutaneously into rats for 7, 14, and 28 days, to determine whether the scaffold is biocompatible. No animal died during the experimental period. With the complete removal of cells from the scaffold, the immunogenic antigens in the acellular tissue are also removed. The acellular ECM scaffold was capable of allowing new tissue formation and was well tolerated by the host with no adverse reactions, indicating that allogeneic decellularized scaffold could be an ideal source for cartilage engineering.

### Examples 2 to 7

### Decellularization of Menisci with Formic Acid at Different Times

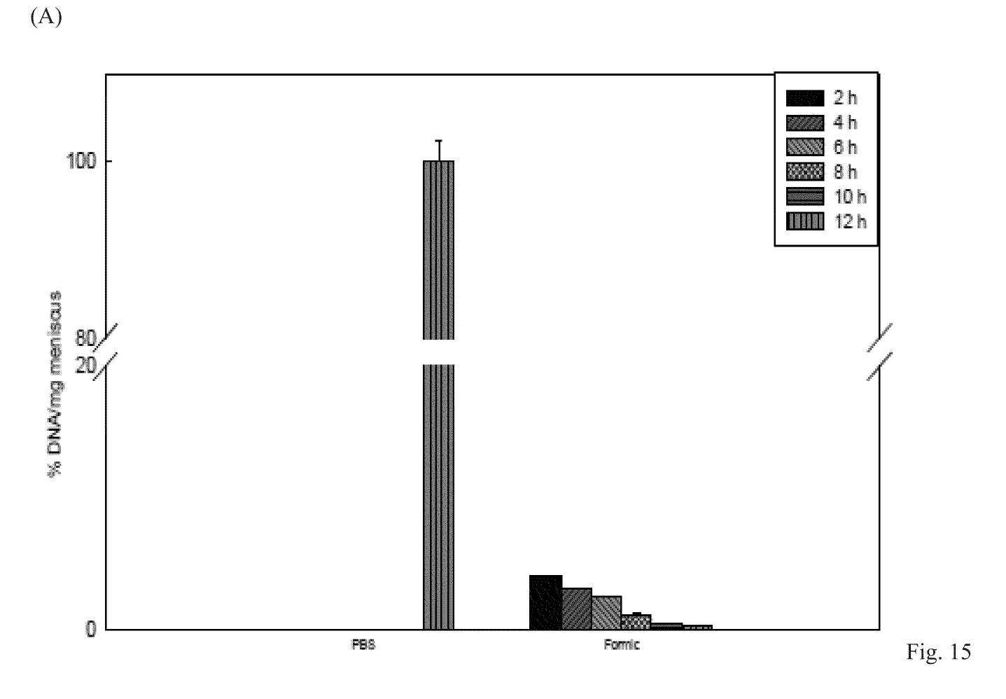

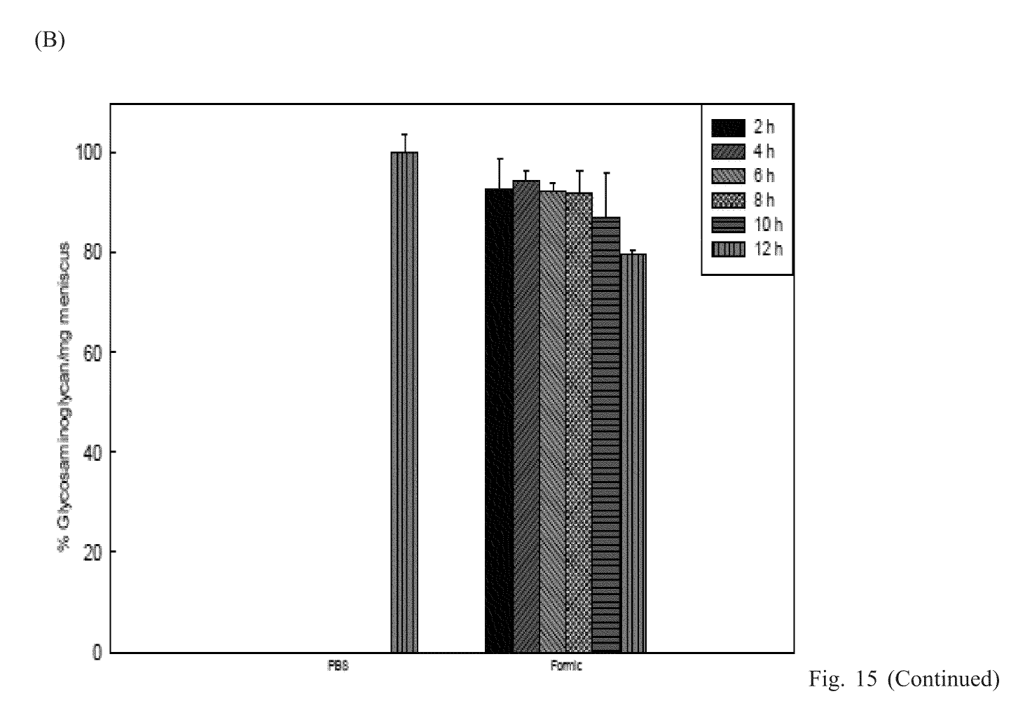

Scaffolds of Examples 2 and 7 were decellularized according to the process described in Example 1 wherein the different times (2, 4, 6, 8, 10 and 12 hours) were used to treat with formic acid as shown in Table 1. Minced menisci were immersed in PBS for 12 h and served as a control group. At predetermined time points, the suspensions were homogenized, dialyzed, and then freeze-dried to fabricate the scaffolds. The success of decellularization was determined by biochemical analysis to compare the amount of DNA and major ECM (GAG and collagen). FIG. 15 shows that the longer treatment of formic acid was, the more efficacy decellularization displayed.

TABLE 1

| Acids | Concentration (%) | Period (h) |
|---|---|---|
| Formic acid | 12.5 | 2, 4, 6, 8, 10, 12 |
| | 25 | |
| | 37.5 | |
| | 50 | |
| | 62.5 | |
| | 75 | |
| | 87.5 | |
| | >99 | |
| Peracetic acid | 0.15 | 2, 4, 6, 8, 10, 12 |
| | 15 | |
| Acetic acid | >99 | 2, 4, 6, 8, 10, 12 |
| Malic acid | 60 | |
| Succinic acid | 5 | |
| Citric acid | 60 | |

### Examples 8 to 14

### Decellularization of Menisci with Formic Acid at Different Concentrations

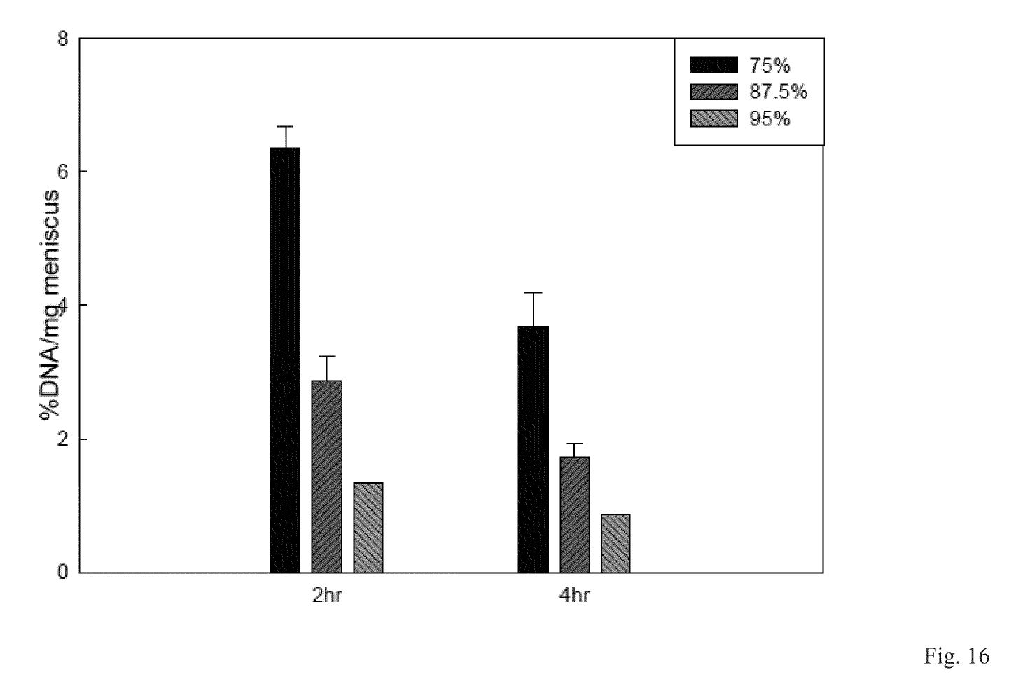

Scaffolds of Examples 8 and 14 were decellularized according to the process described in Example 1 wherein the different concentrations of formic acid (12.5, 25, 37.5, 50, 62.5, 75, 87.5%) were employed for 2 or 4 hours (see Table 1). FIG. 16 shows that the higher concentration of formic acid or the longer treatment period was, the more efficacy decellularization displayed.

### Example 15

### Fabrication of Decellularized Skin Scaffold

Skin was harvested from the adult porcine and washed by PBS. 0.2 g skin was suspended in 10 mL of acetic (>99%), formic (>99%), peracetic (15%) and citric (60%) acids for 24 hours with stirring at room temperature, respectively. Skin was immersed in PBS for 24 hours as a control. After 24 hours, the skin was washed to remove excess acid and cellular debris, and then freeze-dried to fabricate the scaffolds. These scaffolds were sterilized by ethylene oxide and then degassed for determination of DNA.

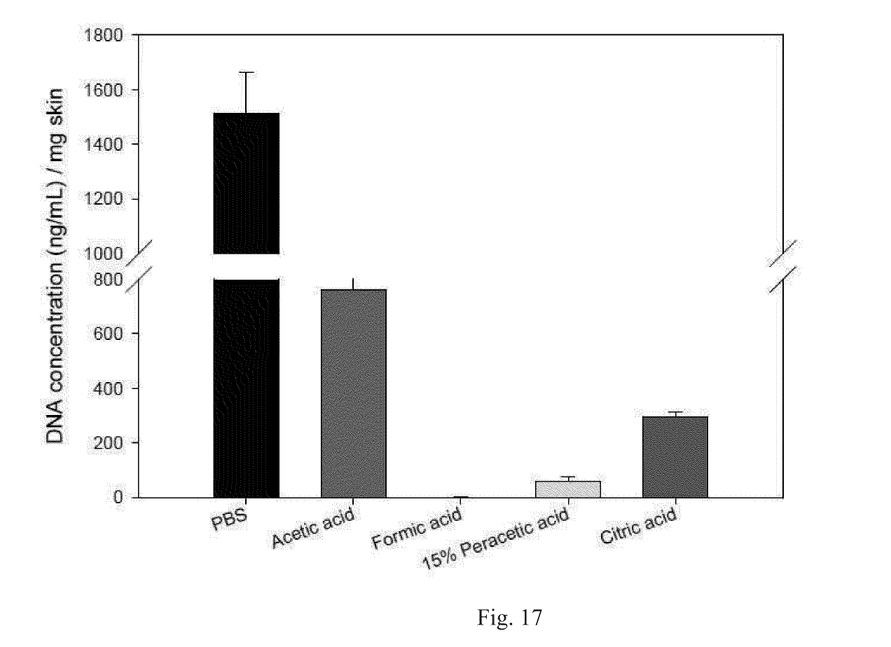

FIG. 17 demonstrates that the use of formic acid can decrease the DNA content to less than 1%; however, the use of acetic acid, and citric acid cannot decrease DNA content to a satisfied level. Although the treatment of peracetic acid was able to decrease DNA dramatically, it eroded the specimen.

### Comparative Examples 1 to 2

Scaffolds of comparative examples 1 to 2 were based on the process described in Example 1 wherein the different concentration of peracetic acid and different treatment period were employed as shown in Table 1. The success of decellularization was determined by biochemical analysis to compare the amount of DNA and major ECM (GAG and collagen).

### Comparative Examples 3 to 6

Scaffolds of comparative examples 3 to 6 were decellularized according to the process described in Example 1 wherein acetic (>99%), malic (60%) succinic (5%) or citric (60%) acids were employed to treat for 2, 4, 6, 8, 10, or 12 hours. We attempted to apply higher concentrations of acid solutions to achieve the best decellularization effect and shorten processing times. The purity of the commercial acid products or their saturated solubility resulted in different concentrations of acids used in the study. The success of decellularization was determined by biochemical analysis to compare the amount of DNA and major ECM (GAG and collagen).

### Scaffolds Fabrication Assay

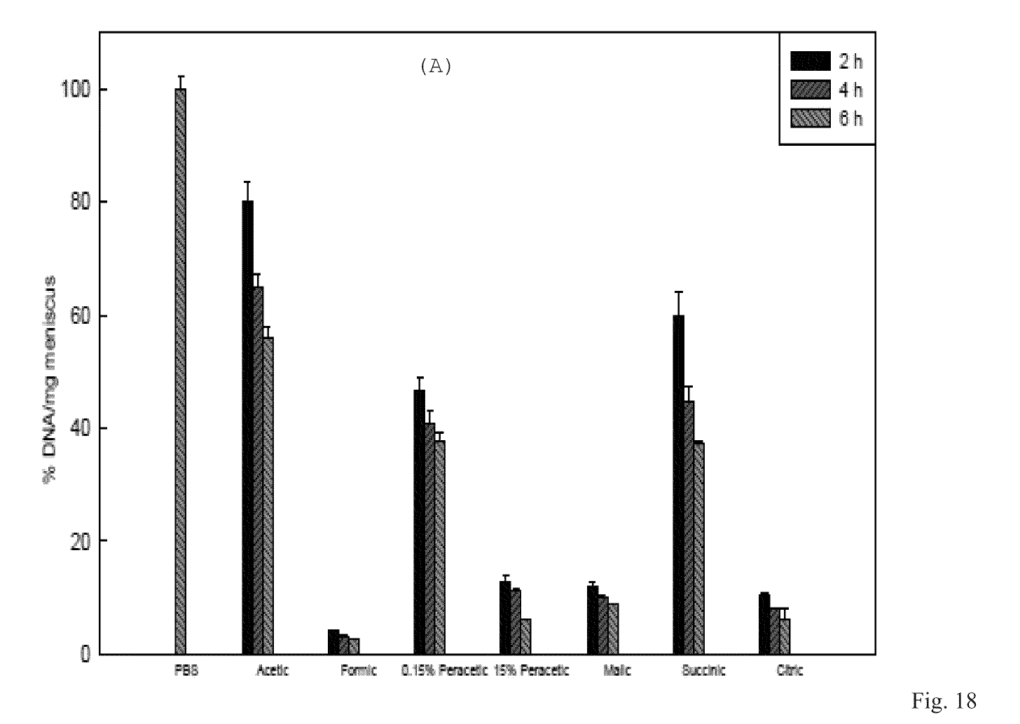

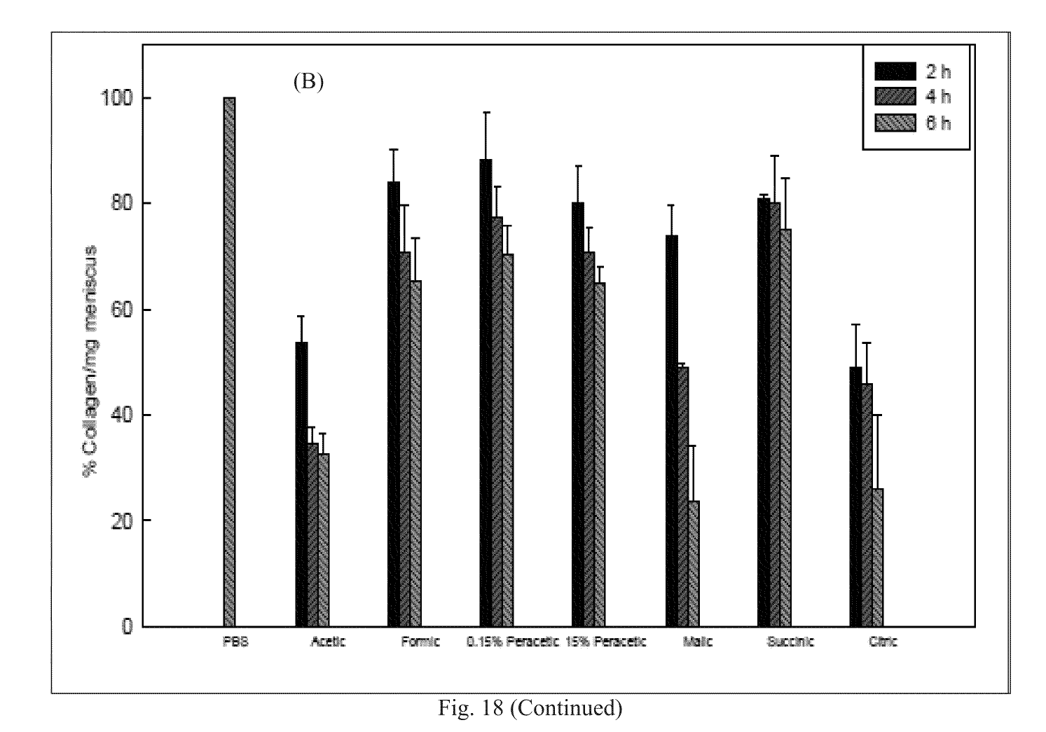

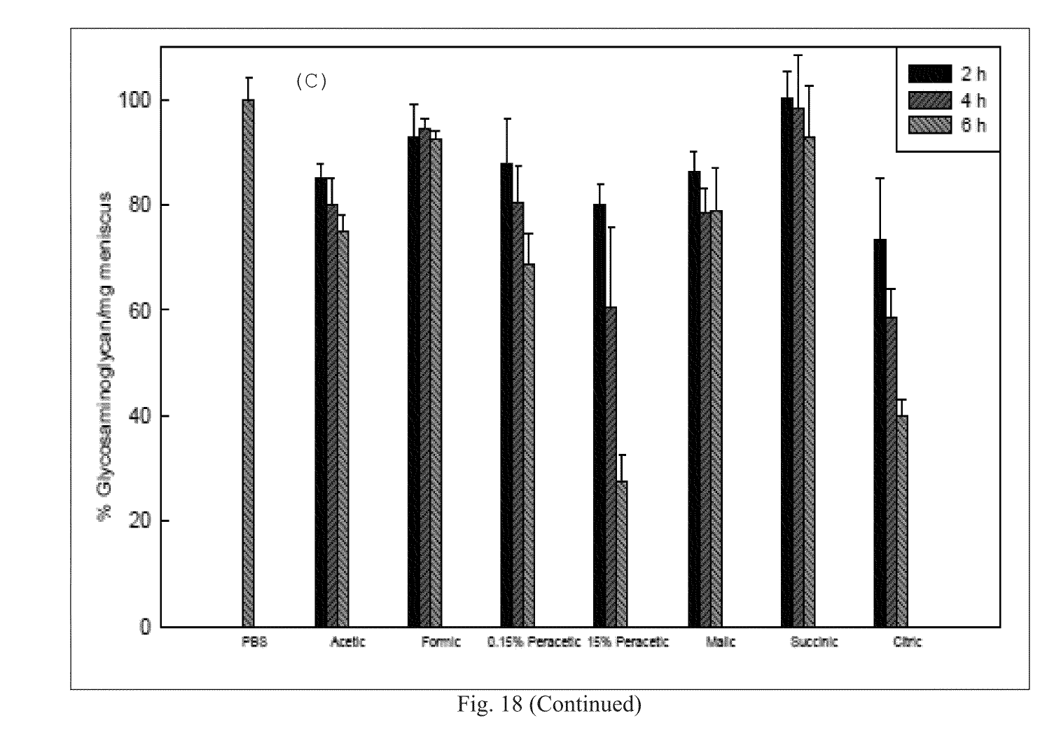

To confirm the removal of cells, DNA quantification was conducted. FIG. 18 (A) shows the relative DNA content compared to control menisci (PBS treatment for 12 hours) after various acid-treatments. Immersion in formic acid solution for 2 hours was able to decrease the amount of DNA to 4.10% (p<0.01), while no obvious influences were found in the menisci treated with acetic, 0.15% peracetic and 5% succinic acids for 2 hours (p=0.095, 0.059, and 0.074, respectively).

FIGS. 18 (B) and (C) display the remaining ratios of major ECM (collagen and GAG) after different treatments. Treatment with formic acid for 2 hours had no significant adverse effect on either GAG or collagen content (p=0.087 and 0.714). Acetic and citric acids caused huge amounts of damage on collagen at 2 hours (p=0.043 and 0.027, respectively), and acetic and 15% peracetic acids enormously decreased the amount of GAG (p=0.038 and <0.01, respectively).

What is claimed is:

1. A method for producing a decellularized biological material, comprising providing an untreated biological material having cells and treating the biological material with a formic acid solution at a concentration effective to remove cellular and nuclear material from the biological material while maintaining higher than about 85% glycosaminoglycan (GAG) compared to the untreated biological material.

2. The method of claim 1, wherein the biological material to be treated by the method of the invention is a tissue and organ.

3. The method of claim 1, wherein the biological material is skin, heart valve, pericardia, blood vessel, spinal cord, trachea, bladder, ligament cartilage, meniscus, disc, bone, dura mater, small intestine submucosa, spinal meninges, kidney, liver, lung, or nerve.

4. The method of claim 1, wherein the concentration of the formic acid in the solution ranges from about 50% (w/w) to about 100% (w/w).

5. The method of claim 1, wherein the decellularized biological material is a decellularized ECM, decellularized tissue, or decellularized organ.

6. The method of claim 1, which further maintains higher than about 75% collagen.

7. The method of claim 1, wherein the formic acid solution can further comprises cosolvents.

8. The method of claim 1, wherein the formic acid solution can further comprises acid solutions.

9. The method of claim 1, wherein the ratio of the biological material to the formic acid is about 1% (w/v) to about 5% (w/v).

10. The method of claim 1, wherein the biological material is treated with a formic acid solution for less than 15 hours.

11. The method of claim 1, wherein the tissue and organ is a heterogeneous or homogeneous, and allogeneic, autologous or xenogenic tissue or organ.

12. The method of claim 1, wherein the content of DNA decreases to less than about 5% or lower compared to the untreated biological material.

13. The method of claim 1, wherein higher than about 90% GAG can be maintained.

14. The method of claim 1, wherein higher than about 80% collagen can be maintained.

15. The method of claim 1, which, before treating the biological material, further comprises a pretreatment step.

16. The method of claim 1, which, before treating the biological material, further comprises a step of physical decellularization, chemical decellularization, or the combination of the physical decellularization and the chemical decellularization.

17. The method of claim 1, which, after treating the biological material, further comprises a washing step.

18. The method of claim 1, which, after treating the biological material, further comprises a step of forming the decellularized biological material as a scaffold by mixing the decellularized biological material with/without porogen, pouring it into a mold for shaping a scaffold and then freeze-drying the shaped scaffold.

19. A decellularized biological material, wherein the contents of DNA and GAG are less than about 5% or lower and higher than about 85%, respectively.

20. The decellularized biological material of claim 19, which further maintains higher than about 75% collagen.

21. The decellularized biological material of claim 19, which is a decellularized ECM, tissue or organ.

22. The decellularized biological material of claim 19, which is combined with a support.

23. The decellularized biological material of claim 19, wherein the support is a pharmaceutical composition, an implant, a scaffold, or a medical device.

24. A method for preparation of an in vitro scaffold culture system, comprising (i) providing a decellularized biological material scaffold of claim 22, (ii) perfusing a population of cells including stem cell, progenitor cells or partially differentiated progenitor cells capable of differentiation, or a population of cells capable of functional maturation to the decellularized biological material scaffold, and (iii) contacting the perfusion decellularized biological material scaffold and the population of cells under conditions and for a period of time that provide for recellularization of the perfusion decellularized biological material scaffold and differentiation and functional maturation of the stem or progenitor cells or functional maturation of the cells in the population.

25. A method for producing a tissue graft, comprising the steps of:
a) providing the decellularized biological material scaffold of claim 22;
b) allowing cells in the organism to infiltrate the decellularized scaffold;
c) incubating the tissue for a time sufficient for the cell to differentiate; and
d) providing a physiologically active substance capable of inducing differentiation of the cell.
